(19) United States
(12) Patent Application Publication
(10) Pub. No.: US 2004/0053831 A1
(43) Pub. Date: Mar. 18, 2004

(54) Veterinary immunisation vectors

(76) Inventors: Michael Steward, Essex (GB); Vivienne Frances Cox, Essex (GB)

Correspondence Address: HELLER EHRMAN WHITE & MCAULIFFE LLP, 1666 K STREET,NW, SUITE 300, WASHINGTON, DC, 20006, US

(21) Appl. No.: 10/398,916

(22) PCT Filed: Oct. 12, 2001
(86) PCT No.: PCT/GB01/04565

(51) Int. Cl. A61K 38/17; C07K 14/705; C07H 21/04

(52) U.S. Cl. 514/12; 530/350; 536/23.2; 435/69.1; 435/320.1; 435/325

(57) ABSTRACT

Variant nucleic acid sequences encoding naturally occurring non human C3d polypeptides with immunostimulatory activity are provided. The variant sequences are non-identical to the naturally occurring DNA sequences encoding the C3d polypeptides by virtue of third base redundancy and/or other variations permissible within an amino acid codon. The variant nucleic acid sequences can be included in veterinary immunisation vectors fused to one or more sequences encoding an antigen to enhance the immunogenicity of the antigen in the host.

This invention relates to novel C3d polypeptides, or fragments thereof, and to nucleic acid sequences encoding such polypeptides or fragments. The invention also relates to genetic constructs comprising genetic sequences encoding species-specific polypeptides designed to enhance the immunogenicity of antigens in non-human species and to methods for the generation of such constructs.

The invention further relates to the use of variant nucleic acid sequences to encode species-specific C3d polypeptides which, when used to express tandem arrays of the polypeptide show enhanced stability, leading to high level expression in eukaryotic and prokaryotic cell expression systems. When incorporated into a DNA immunization vector or a recombinant live organism vaccine, such sequences have reduced risk of undergoing homologous recombination with genomic DNA compared to wild-type sequences, thus reducing the risk of potentially damaging integration events.

A species-specific polypeptide linked to an antigen, or nucleic acid encoding the same may be administered as part of a prophylactic or therapeutic vaccine formulation to any non-human vertebrate (the host species), or administered with the intention of raising specific antibodies to the antigen in the host species. Such antigens may be derived from any organism including the host species. The species-specific polypeptides comprise or consist of tandem arrays of a polypeptide which occurs naturally in the host species and which has immunostimulatory properties. Examples of such polypeptides include polypeptides derived from the complement system, as described below. Such tandem arrays, when linked to an antigen may enhance humoral responses to the antigen by several orders of magnitude.

A number of naturally occurring immune modulators, such as cytokines, have been proposed for inclusion into DNA immunization vectors to be expressed concurrently with the antigen (reviewed by Leitner et al., 1999 Vaccine 18: 765-77). The use of naked DNA as an immunogen has raised concerns about the potential for its integration into the genome of the host species and the possibility of insertional mutagenesis resulting in the inactivation of tumor suppressor genes or the activation of oncogenes (reviewed by Nicholls et al., 1995 Ann N Y Acad Sci 772: 30-9). Such concerns apply equally to recombinant live organisms used as vaccines, many of which undergo rounds of self-replication in the host species. Although the studies reviewed by Nicholls et al., (1995) have shown integration to be a low frequency occurrence with plasmids containing sequences unrelated to the host species the inclusion of genes derived from the genome of the host species increases this risk significantly.

The complement system consists of a set of serum proteins that are important in the response of the immune system to foreign antigens. The complement system becomes activated when its primary components are cleaved and the products, alone or with other proteins, activate additional complement proteins resulting in a proteolytic cascade. Activation of the complement system leads to a variety of responses including increased vascular permeability, chemotaxis of phagocytic cells, activation of inflammatory cells, opsonisation of foreign particles, direct killing of cells and tissue damage. Activation of the complement system may be triggered by antigen-antibody complexes (the classical pathway) or a normal slow activation may be amplified in the presence of cell walls of invading organisms such as bacteria and viruses (the alternative pathway).

The complement system interacts with the cellular immune system through a specific pathway involving C3, a protein central to both classical and alternative pathways. The proteolytic activation of C3 gives rise to a large fragment (C3b) and exposes a chemically reactive internal thiolester linkage which can react covalently with external nucleophiles such as the cell surface proteins of invading organisms or foreign cells. As a result, the potential antigen is ‘tagged’ with C3b and remains attached to that protein as it undergoes further proteolysis to iC3b and C3d,g. The latter fragments are, respectively, ligands for the complement receptors CR3 and CR2. Thus the labelling of antigen by C3b can result in a targeting mechanism for cells of the immune system bearing these receptors.

That such targeting is important for augmentation of the immune response is first shown by experiments in which mice were depleted of circulating C3 and then challenged with an antigen (sheep erythrocytes). Removal of C3 reduced the antibody response to this antigen. (M. B. Pepys, J.Exp.Med, 140, 126-145, 1974). The role of C3 was confirmed by studies in animals genetically deficient in either C3 or the upstream components of the complement cascade which generate C3b, i.e. C2 and C4, (J. M. Ahearn & D. T. Fearon, Adv.Immunol. 46, 183-219, 1989). More recently, it has been shown that linear conjugation of a model antigen with more than two copies of the murine C3d fragment sequence resulted in a very large (1000-10000-fold) increase in antibody response in mice compared with unmodified antigen controls (P. W. Dempsey et al, Science, 271: 348-350, 1996; WO96/17625, PCT/GB95/02851). The increase could be produced without the use of conventional adjuvants such as Freund's complete adjuvant. The mechanism of this remarkable effect was demonstrated to be high-affinity binding of the multivalent C3d construct to CR2 on B-cells, followed by co-ligation of CR2 with another B-cell membrane protein, CD19, and with membrane-bound immunoglobulin to generate a signal to the B-cell nucleus.

In the experiments of Dempsey et al, (1996) the unmodified antigen control and linear fusions with one or two C3d domains were prepared by transfection of the appropriate coding plasmids into L cells followed by the selection of high-expressing clones. The most immunogenic construct, that with three C3d units, had to be expressed transiently in COS cells and this procedure gave a very poor yield of the fusion protein. In part, the low yield could be attributed to the generation of species containing the antigen but with lower molecular weights, corresponding to fewer than three C3d units. It was unclear from the published work of Dempsey et al whether the latter molecules originated by proteolysis of the three-C3d construct or whether they were due to a recombination event in vivo.

Using another expression system but the same C3d constructs as Dempsey et al, we obtained evidence that the generation of molecules with <3 C3d units from DNA encoding 3× C3d repeats is due to loss of one or more C3d units by homologous recombination and not due to post-translational processing (see WO99/35260) and described methods for the generation and selection of stable variant genes resistant to homologous recombination.

The present invention is defined in the appended independent claims. Preferred features of the invention are specified in the subclaims.

This invention may be used in any context where a nucleic acid sequence is included in a medicament where the sequence of the nucleic acid is homologous to a sequence in the genome of the recipient human or animal host. These may be used in the context of gene therapy, therapeutic or prophylactic vaccination of other therapeutic strategies in which nucleic acid forms part of the medicament. It is particularly useful for, but is not restricted to, DNA immunization vectors encoding proteins with immunopotentiating properties derived from the complement system.

Preferred embodiments of this invention related specifically to an immunostimulatory component of the complement system, and the use of species-specific components in veterinary vaccines for non-human vertebrate species or to raise antibodies in non-human vertebrate species.

The present invention provides:

i) a general method for cloning C3d from non-human vertebrates and the construction of variant DNA sequences encoding identical C3d units which can be ligated in tandem with each other with or without the native (wild-type) C3d DNA sequence and may be stably maintained in prokaryotic and eukaryotic expression vectors to produce oligomers of at least two or three copies of species-specific C3d at commercially viable levels; and

ii) the use of the oligomers in combination with antigens as vaccines or the nucleic acid sequence encoding the oligomers in DNA immunization vectors or in recombinant live organisms with reduced capacity for homologous integration into host genomic DNA.

The present invention also provides:

1. Novel C3d cDNA sequences from non-human vertebrate species, and methods of cloning such sequences.

2. Novel synthetic DNA sequences encoding oligomers of species-specific C3d where the polypeptide sequence of each unit of the C3d is identical, but the DNA encoding each unit is different, and methods of constructing such sequences.

3. High-level expression of oligomers of species-specific C3d in prokaryotic and eukaryotic systems and maintenance of stable recombinant expression vector stocks.

4. Use of variant C3d genes encoding species-specific C3d fused to antigen in a DNA immunization vector.

5. Use of variant C3d genes encoding species-specific C3d fused to antigen in a recombinant live organism.

The present invention also provides a process for preparing an oligomeric polypeptide in vitro or in vivo comprising: constructing an expression vector, which may be a DNA vector or a recombinant live organism encoding the oligomeric polypeptide; introducing the expression vector into a recombinant host cell in vitro or a host organism in vivo; and culturing the recombinant host cell or host organism under conditions for expression of the polypeptide.

The process may further comprise amplifying species-specific nucleic acid encoding a C3d polypeptide from tissue derived from a vertebrate species. The process may further comprise recovering the polypeptide.

The present invention also provides a process for preparing an nucleic acid encoding a C3d polypeptide which comprises: amplifying species-specific nucleic acid encoding a C3d polypeptide from tissue derived from a vertebrate species; preparing a replicable expression vector from the amplified nucleic acid which encodes the C3d polypeptide; transforming a host cell with the vector; culturing the transformed host cell under conditions for replication of the expression vector; and recovering the expression vector in a form suitable for DNA immunization.

There is also provided a linear DNA concatameter encoding the oligomeric polypeptide.

Processes of the invention may be performed using conventional recombinant techniques such as described in Sambrook et al., Molecular Cloning: A laboratory manual 2nd Edition. Cold Spring Harbor Laboratory Press (1989) and DNA Cloning vols I, II and III (D. M. Glover ed., IRL Press Ltd).

The invention also provides a process for preparing the linear DNA concatamer by condensing appropriate mono-, di- or oligomeric nucleotide units.

The preparation may be carried out chemically, enzymatically, or by a combination of the two methods, in vitro or in vivo as appropriate. Thus, the linear DNA concatamer may be prepared by the enzymatic ligation of appropriate DNA fragments, by conventional methods such as those described by D. M. Roberts et al., in Biochemistry 1985, 24, 5090-5098.

The DNA fragments may be obtained by digestion of DNA containing the required sequences of nucleotides with appropriate restriction enzymes, by chemical synthesis, by enzymatic polymerisation, or by a combination of these methods.

Digestion with restriction enzymes may be performed in an appropriate buffer at a temperature of 20°-70° C., generally in a volume of 50 μl or less with 0.1-10 μg DNA. Enzymatic polymerisation of DNA may be carried out in vitro using a DNA polymerase such as DNA polymerase 1 (Klenow fragment) in an appropriate buffer containing the nucleoside triphosphates dATP, dCTP, dGTP and dTTP as required at a temperature of 10°-37° C., generally in a volume of 50 μl or less. Enzymatic ligation of DNA fragments may be carried out using a DNA ligase such as T4 DNA ligase in an appropriate buffer at a temperature of 4° C. to 37° C., generally in a volume of 50 μl or less.

The chemical synthesis of the linear DNA concatamer or fragments may be carried out by conventional phosphotriester, phosphite or phosphoramidite chemistry, using solid phase techniques such as those described in ‘Chemical and Enzymatic Synthesis of Gene Fragments—A Laboratory Manual’ (ed. H. G. Gassen and A. Lang), Verlag Chemie, Weinheim (1982), or in other scientific publications, for example M. J. Gait, H. W. D. Matthes M. Singh, B. S. Sproat and R. C. Titmas, Nucleic Acids Research, 1982, 10, 6243; B. S. Sproat and W. Bannwarth, Tetrahedron Letters, 1983, 24, 5771; M. D. Matteucci and M. H. Caruthers, Tetrahedron Letters, 1980, 21, 719; M. D. Matteucci and M. H. Caruthers, Journal of the American Chemical Society, 1981, 103, 3185;S. P. Adams et al., Journal of the American Chemical Society, 1983, 105, 661; N. D. Sinha, J. Biernat, J. McMannus and H. Koester, Nucleic Acids Research, 1984, 12, 4539; and H. W. D. Matthes et al., EMBO Journal, 1984, 3, 801. Preferably an automated DNA synthesiser (for example, Applied Biosystems 381A Synthesiser) is employed.

The linear DNA concatamer is preferably prepared by ligating two or more DNA molecules which together comprise a DNA sequence encoding the oligomeric polypeptide. The DNA molecules may be obtained by digestion with suitable restriction enzymes of vectors carrying the required coding sequences.

The precise structure of the DNA molecules and the way in which they are obtained depends upon the structure of the desired product. A linear DNA concatamer encoding the oligomeric polypeptide may be constructed using a variety of methods including chemical synthesis of DNA oligonucleotides, enzymatic polymerisation, restriction enzyme digestion and ligation.

Expression of the oligomeric polypeptide encoded by the linear DNA concatamer in a recombinant host cell or in vivo by a recipient of a DNA immunisation vector may be carried out by means of a replicable expression vector capable, in the host cell or in vivo, of expressing the polypeptide from the DNA polymer.

The replicable expression vector may be prepared by cleaving a vector compatible with the host cell to provide a linear DNA segment having an intact replicon, and combining said linear segment with one or more DNA molecules which, together with said linear segment, encode the polypeptide, under ligating conditions.

Ligation of the linear segment and more than one DNA molecule may be carried out simultaneously or sequentially as desired. Thus, the linear DNA concatamer may be preformed or formed during the construction of the vector, as desired.

The choice of vector will be determined in part by the host cell, which may be prokaryotic, such as E. coli, mammalian, such as mouse C127, mouse myeloma, Chinese hamster ovary, or other eukaryotic (fungi e.g. filamentous fungi or unicellular yeast or an insect cell such as Drosophila or Spodoptera). The host cell may also be in a transgenic animal or a human or animal recipient of a DNA immunization vector. Suitable vectors include plasmids, bacteriophages, cosmids and recombinant viruses derived from, for example, baculoviruses, vaccinia, adenovirus and herpesvirus.

The linear DNA concatamer may be assembled into vectors designed for isolation of stable transformed mammalian cell lines expressing the fragment e.g. bovine papillomavirus vectors in mouse C127 cells, or amplified vectors in Chinese hamster ovary cells (DNA Cloning Vol. II D. M. Glover ed. IRL Press 1985; Kaufman, R. J. et al.. Molecular and Cellular Biology 5, 1750-1759, 1985; Pavlakis G. N. and Hamer, D. H. Proceedings of the National Academy of Sciences (USA) 80, 397-401, 1983; Goeddel, D. V. et al. European Patent Application No. 0093619, 1983).

The preparation of the replicable expression vector may be carried out conventionally with appropriate enzymes for restriction, polymerisation and ligation of the DNA, by procedures described in, for example, Sambrook et al., cited above. Polymerisation and ligation may be performed as described above for the preparation of the linear DNA concatamer. Digestion with restriction enzymes may be performed in an appropriate buffer at a temperature of 20°-70° C., generally in a volume of 50 μl or less with 0.1-10 μg DNA.

A recombinant host cell may be prepared, in accordance with the invention, by transforming a host cell with a replicable expression vector of the invention under transforming conditions. Suitable transforming conditions are conventional and are described in, for example, Sambrook et al., cited above, or “DNA Cloning” Vol. II, D. M. Glover ed., IRL Press Ltd, 1985.

The choice of transforming conditions is determined by the host cell. Thus, a bacterial host such as E. coli, may be treated with a solution of CaCl₂ (Cohen et al., Proc. Nat. Acad. Sci., 1973, 69, 2110) or with a solution comprising a mixture of RbCl, MnCl₂, potassium acetate and glycerol, and then with 3-[N-morpholino]-propane-sulphonic acid, RbCl and glycerol or by electroporation as for example described by Bio-Rad Laboratories, Richmond, Calif., USA, manufacturers of an electroporator. Eukaryotic cells in culture may be transformed by calcium co-precipitation of the vector DNA onto the cells or by using cationic liposomes.

DNA immunization vectors may be administered as naked DNA or contained within a viral particle by injection or by other means of delivery including aqueous or non-aqueous formulations via transdermal or mucosal routes.

The invention also provides a host cell transformed with a replicable expression vector of the invention.

Culturing the transformed host cell under conditions for expression of the linear DNA concatamer may be carried out conventionally, as described in, for example, Sambrook et al., and “DNA Cloning” cited above. Thus, preferably the cell is supplied with nutrient and cultured at a temperature below 45° C.

An oligomeric polypeptide of the invention may be recovered by conventional methods. Thus, where the host cell is bacterial such as E. coli and the oligomeric polypeptide is expressed intracellularly, it may be lysed physically, chemically or enzymatically and the oligomeric polypeptide isolated from the resulting lysate. Where the host cell is eukaryotic, the oligomeric polypeptide may be isolated from the nutrient medium. Where the host cell is in a transgenic animal the polypeptide may be recovered from the natural secretory pathways (e.g. where the polypeptide is secreted in the milk of a female transgenic animal). Where the host cell is in a human or animal recipient of a DNA immunization vector or gene therapy vector the oligomeric polypeptide would not normally be recovered, but may be detected in tissues for the purpose of evaluating the utility of the delivery system.

WO99/35260 describes methods for purification and refolding (where required) of protein products expressed in prokaryotic and eukaryotic systems and its contents are incorporated herein by reference.

Nucleic acid of the invention may encode an additional cysteine residue which can be expressed at the carboxy-terminus or other location within a polypeptide of the invention. The utility and post-translational modification of the carboxy-terminal cysteine is described in WO99/35260.

The use of insect cells infected with recombinant baculovirus encoding the oligomeric polypeptide is a preferred general method for preparing complex proteins, particularly an oligomeric polypeptide of the invention encoding C3d oligomers or fusions of the C3d oligomers with an antigen.

The use of DNA immunization vectors or recombinant live organisms is an alternative general method for delivery of an oligomeric polypeptide encoding C3d oligomers fused to antigen in vivo as an immunogen for prophylactic or therapeutic purposes.

### GENERAL METHODS USED IN EXAMPLES

(i) DNA Cleavage

Cleavage of DNA by restriction endonucleases was carried out according to the manufacturer's instructions using supplied buffers (New England Biolabs (U.K.) Ltd., Herts. or Promega Ltd., Hants, UK). Double digests were carried out simultaneously if the buffer conditions were suitable for both enzymes. Otherwise double digests were carried out sequentially where the enzyme requiring the lowest salt condition was added first to the digest. Once the digest was complete the salt concentration was altered and the second enzyme added.

(ii) DNA Ligation

Ligations were carried out using T4 DNA ligase purchased from Promega or New England Biolabs as described in Sambrook et al, (1989) Molecular Cloning: A Laboratory Manual 2nd Edition, Cold Spring Harbor Laboratory Press.

(iii) Plasmid Isolation

Plasmids were isolated using Wizard™ Plus Minipreps (Promega) or Qiex mini or midi kits and Qiagen Plasmid Maxi kit (QIAGEN, Surrey) according to the manufacturer's instructions.

(iv) DNA Fragment Isolation

DNA fragments were excised from agarose gels and DNA extracted using the QIAEX gel extraction kit or Qiaquick (QIAGEN, Surrey, UK), or GeneClean, or GeneClean Spin Kit or MERmaid Kit, or MERmaid Spin Kit (Bio 101 Inc, CA. USA) gel extraction kits according to the manufacturer's instructions.

(v) Introduction of DNA Into E. coli

Plasmids were transformed into competent E. coli BL21 (DE3) or XL1-blue strains (Studier and Moffat, (1986), J. Mol. Biol. 189:113). The E. coli strains were purchased as a frozen competent cultures from Stratagene (Cambridge, UK).

(vi) DNA Sequencing

The sequences were analysed by a Perkin Elmer ABI Prism 373 DNA Sequencer. This is an electrophoretic technique using 36 cm×0.2 mm 4% acrylamide gels, the fluorescently labeled DNA fragments being detected by a charge coupled device camera according to the manufacturer's instructions.

(vii) Production of Oligonucleotides and Synthetic Genes

Oligonucleotides and synthetic genes were purchased from Cruachem, Glasgow, UK or from Sigma-Genosys, Cambridge, UK.

(viii) Generation of Baculovirus Vectors

Plasmids described in this invention having the prefix pBP or pBAC are used to generate baculovirus vectors and express the encoded recombinant polypeptides by the following methods (Sections (viii) to (x)). Purified plasmid DNA was used to generate recombinant baculoviruses using the kits ‘The BacPak Baculovirus Expression System’ (Clontech, CA, USA) or ‘BacVector 3000’ (Invitrogen) according to the manufacturers' protocols. The insect cell line Sf9 (ATCC) was grown in Sf900II medium (Gibco) at certain times supplemented with foetal calf serum (Gibco, Paisley, UK). Cells were transfected with the linearised baculovirus DNA (supplied in the kits) and the purified plasmid. Plaque assays (see method below) were carried out on culture supernatants and a series of ten-fold dilutions thereof to allow isolation of single plaques. Plaques were picked using glass Pasteur pipettes and transferred into 0.5 ml aliquots of growth medium. This is the primary seed stock.

(ix) Plaque Assay of Baculoviruses

1×10⁶ Sf9 cells were seeded as monolayer cultures in 30 mm plates and left to attach for at least 30 minutes. The medium was poured off and virus inoculum in 100 μl growth medium was dripped onto the surface of the monolayer. The plates were incubated for 30 minutes at room temperature, occasionally tilting the plates to prevent the monolayer from drying out. The monolayer was overlaid with a mixture of 1 ml growth medium and 3% (w/v) “Seaplaque” agarose (FMC, ME) warmed to 37° C. and gently swirled to mix in the inoculum. Once set a liquid overlay of 1 ml growth medium was applied. The plates were incubated in a humid environment for 3-5 days.

Visualisation of plaques was achieved by addition to the liquid overlay 1 ml phosphate buffered saline (PBS) containing neutral red solution at 0.1% (w/v) from a stock solution of 1% (w/v) (Sigma, Dorset,UK). Plaques were visible as circular regions devoid of stain up to 3 mm in diameter.

(x) Scale-Up of Baculovirus Vectors and Protein Expression

200 μl of the primary seed stock was used to infect 1×10⁶ Sf9 monolayer cell cultures in 30 mm plates. The seed stock was dripped onto the monolayer and incubated for 20 minutes at room temperature, and then overlaid with 1 ml growth medium. The plates were incubated at 27° C. in a humid environment for 3-5 days. The supernatant from these cultures is Passage 1 virus stock. The virus titre was determined by plaque assay and further scale up was achieved by infection of monolayer cultures or suspension cultures at a multiplicity of infection (moi) of 0.1. Virus stock were passaged a maximum of six times to minimise the emergence of defective virus.

Expression of recombinant proteins was achieved by infection of monolayer or suspension cultures in growth medium with or without foetal calf serum (FCS). Where FCS was omitted cells conditioned to growth in the absence of FCS were used. Virus stocks between passage 1 and 6 were used to infect cultures at a moi of >5 per cell. Typically, infected cultures were harvested 72 hours post infection and recombinant proteins isolated either from the supernatants or the cells.

(xiv) Protein Purification

A number of standard chromatographic techniques can be used to isolate the C3d-containing proteins, e.g. such methods as ion-exchange and hydrophobic interaction matrixes chromatography utilising the appropriate buffer systems and gradient to purify the target proteins. The properties of the C3d containing fusion polypeptides will vary depending on the nature of the fusion protein. Examples of methods employed are described in WO99/35260.

(xv) Sodium Dodecyl Sulphate Polyacrylamide Gel Electrophoresis (SDS-PAGE)

SDS-PAGE was carried out generally using the Novex system (Novex GmbH, Heidleburg) according to the manufacturer's instructions. Pre-packed gels of Tris/glycine a 4-20% acrylamide gradient were usually used. Samples for electrophoresis, including protein molecular weight standards (for example LMW Kit, Pharmacia, Sweden or Novex Mark 12, Novex, Germany) were usually diluted in 1% (w/v) SDS—containing buffer (with or without 5% (v/v) 2-mercaptoethanol), and left at room temperature for 5 to 30 min before application to the gel.

(xvi) Immunoblotting

(a) Dot Blot

Immobilon membranes (Millipore, Middlesex, UK) were activated by immersion in methanol for 20 seconds and then washed in PBS for five minutes. The membrane was placed into a vacuum manifold Dot Blotter (Bio-Rad Laboratories, Watford, UK). Crude extracts from cells or culture supernatants were transferred onto the membrane by applying a vacuum and washed through with PBS. Without allowing the membrane to dry out, the Dot Blotter was dismantled and the membrane removed.

(b) Western Blotting

Samples of cell extracts and purified proteins were separated on SDS-PAGE as described in Section (xv). The Immobilon membrane was prepared for use as in (a) above. The gel and the membrane were assembled in the Semi-Dry Transfer Cell (Trans-Blot SD, Bio-Rad Laboratories) with the Immobilon membrane towards the anode and the SDS-PAGE gel on the cathode side. Between the cathode and the gel were placed 3 sheets of Whatman 3M filter paper cut to the size of the gel pre-soaked in a solution of 192 mM 6-amino-n-caproic acid, 25 mM Tris pH 9.4 containing 10% (v/v) methanol. Between the anode and the membrane were placed two sheets of Whatman 3M filter paper cut to the size of the gel and soaked in 0.3M -Tris pH 10.4 containing 10% (v/v) methanol next to the anode and on this was laid a further sheet of Whatman 3M filter paper pre-soaked in 25 mM Tris pH 10.4 containing 10% (v/v) methanol.

The whole-assembled gel assembly was constructed to ensure the exclusion of air pockets. The proteins were transferred from the SDS-PAGE to the Immobilon membrane by passing 200 mA current through the assembly for 30 minutes.

(c) Immunoprobing of Dot Blot and Western Membranes

The membranes were blocked by incubating the membrane for 1 h at room temperature in 50 ml of 10 mM phosphate buffer pH 7.4 containing 150 mM NaCl, 0.02% (w/v) Ficoll 400, 0.02% (w/v) polyvinylpyrolidine and 0.1% (w/v) bovine serum albumin (BSA). The appropriate primary antibody was diluted to its working concentration in antibody diluent, 20 mM sodium phosphate buffer pH 7.4 containing 0.3M NaCl, 0.5% (v/v) Tween-80 and 1.0% (w/v) BSA. The membrane was incubated for 2 h at room temperature in 50 ml of this solution and subsequently washed three times for 2 minutes in washing buffer, 20 mM sodium phosphate pH 7.4 containing 0.3M NaCl and 0.5% (v/v) Tween-80. The membrane was then transferred to 50 ml of antibody diluent buffer containing a suitable dilution of the species specific antibody labelled with the appropriate label, e.g. biotin, horse radish peroxidase (HRP), for the development process chosen and incubated for 2 h at room temperature. The membrane was then washed in washing buffer as described above. Finally, the blot was developed according to the manufacturer's instructions.

The appropriate dilution of antibody for both the primary and secondary antibodies refers to the dilution that minimises unwanted background noise without affecting detection of the chosen antigen using the development system chosen. This dilution is determined empirically for each antibody.

(xvii) Gene Sequences

The sequence of wild-type human C3d is available on public databases under accession number K02765. Other published C3d sequences include mouse Mus musculus (K02782), rat Rattus norvegicus (X52477), guinea pig Cavia porcellus (M34054), rabbit Oryctolagus cuniculus (M32434), sheep Ovis aries (AF038130), chicken Gallus gallus (U16848), cobra Naja naja (L02365), lamprey Lampetra japonica (D10087) , toad Xenopus laevis (U19253), carp Cyprinus carpio (AB016210), trout Oncrhynchus mykiss (L24433) and sea urchin Strongylocentrotus purpuratus (AFO25526).

Variant gene sequences for human and mouse C3d are given in WO99/35260. The sequence of all novel species-specific C3d sequences and variant DNA sequences encoding concatamers of the same polypeptide are described in the following examples and in the appendices.

### EXAMPLES

1. Cloning of C3d From Non-Human Vertebrate Tissue Using Degenerate Primers

1.1 Primer Design

The degenerate primers used to clone the species-specific C3d sequences were designed by alignment of existing C3 protein sequences from human, mouse, rat, and guinea pig. Regions of amino acid conservation within and flanking the C3d region, where low codon redundancy was prominent were selected by eye, and oligonucleotides for RT-PCR designed to incorporate redundant bases where necessary

| (FARM | TGY GGR GAR CAG AAC ATG ATY GGC ATG | SEQID1 |
|---|---|---|
| 1) | | |
| (FARM | CCG TAG TAT CTY ASN TCR TTG AGC CA | SEQID2 |
| 2) | | |
| (FARM | GGA GTC TTC GAG GAG AAT GGG CC | SEQID3 |
| 3) | | |
| (FARM | GTG TGT CWG GRR CRA AGC CRG TCA TCA T | SEQID4 |
| 4) | | |
| (FARM | GTR ATG CAG GAC TTC TTC ATY GAC CTG | SEQID5 |
| 5) | | |
| (FARM | GGC TGT CAG GGA CAC GTC TTT CTC | SEQID6 |
| 6) | | |
| (FARM | GCA AGG GAC CCC MGT GGC CCA GAT G | SEQID7 |
| 7) | | |
| (FARM | GYC ACC ACC GAC AAK GTG CCT TG | SEQID8 |
| 8) | | |

R = G/A, Y = C/T, W = A/T, S = G/C, K = G/T,
M = A/C, N = A/C/G/T.

1.2 Reverse Transcription-PCR

Total RNA was purified from liver or other tissue samples of horse (Equus caballus), pig (Sus scrofa), marmoset (Callithrix sp.), cat (Felis catus) and dog (Canis familiaris) by the acid-guanidinium thiocyanate-phenol chloroform extraction technique of Chomczynski and Sacchi (Anal. Biochem 162: 156-159 (1987)). RNA extracted from bovine liver (Bos taurus) was obtained commercially (Clontech). Approximately 3 ug of RNA was used in the RT reaction using the reverse transcription system from Promega. Reverse transcription was primed with 40 pmol of anti-mRNA sense primer, (ie. any of the even-numbered primers).

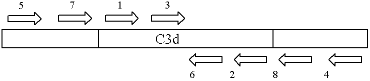

In some cases a single round PCR was sufficient to generate a positive product, whereas on others nested PCR was necessary. For example an outer PCR with primers FARM 4 and 5 was followed by inner PCR with primers FARM 6 &7 and 3 &8, thus covering the entire C3d region. PCR conditions were typically 95° C. 30 sec, 54° C. 30 sec, 72° C. 60 sec, x35 cycles.

1.3 Subcloning and Sequencing of Novel C3d Clones From Cow, Pig, Horse and Marmoset, Cat and Dog

PCR products derived from horse (Equus Caballus), pig (Sus scrofa), marmoset (Callithrix sp.), cat (Felis catus) and dog (Canis familiaris) and cow (Bos taurus) were subcloned into pU57/T (MBI Fermentas) and a minimum of three clones covering any region of C3d were fully sequenced and aligned using the SeqMan module of the DNAStar software package. The amino acid and nucleic acid sequences are given in SEQID 9-18

1.4 Design of Variant Genes to Prevent Homologous Recombination

For each native sequence published or cloned de novo variant genes may be designed which encode the same amino acid sequence but which contain a large number of silent mutations. These sequences may be cloned in isolation or in tandem with the native sequence and are resistant to homologous recombination. These sequences allow expression of concatamers of C3d from DNA which would otherwise undergo homologous recombination. In addition when used in DNA immunization vectors, or in vectors derived from live organisms with the intention of raising antibodies to antigens cloned in tandem to the C3d, the variant genes are resistant to homologous recombination with the native C3d present in the genome of the host species. Examples of variant genes for pig, cow and dog are given in SEQID 19 to 24.

### Appendix 1

### Sequences Described in the Text

SEQID1: Nucleotide sequence of PCR primer FARM 1

TGYGGRGARCAGAACATGATYGGCATG

SEQID2: Nucleotide sequence of PCR primer FARM 2

CCGTAGTATCTYASNTCRTTGAGCCA

SEQID3: Nucleotide sequence of PCR primer FARM 3

GGAGTCTTCGAGGAGAATGGGCC

SEQID4: Nucleotide sequence of PCR primer FARM 4

GTGTGTCWGGRRCRAAGCCRGTCATCAT

SEQID5: Nucleotide sequence of PCR primer FARM 5

GTRATGCAGGACTTCTTCATYGACCTG

SEQID6: Nucleotide sequence of PCR primer FARM 6

GGCTGTCAGGGACACGTCTTTCTC

SEQID7: Nucleotide sequence of PCR primer FARM 7

GCA AGG GAC CCC MGT GGC CCA GAT G

SEQID8: Nucleotide sequence of PCR primer FARM 8

GYC ACC ACC GAC AAK GTG CCT TG

R=G/A, Y=C/T, W=A/T, S=G/C, K=G/T, M=A/C, N=A/C/G/T.

SEQID9: Amino acid sequence of C3d from cow

| | |
|---|---|
| 1 | His Leu Ile Gln Thr Pro Ser Gly Cys Gly |
| 11 | Glu Gln Asn Met Ile Gly Met Thr Pro Thr |
| 21 | Val Ile Ala Val His Tyr Leu Asp Ser Thr |
| 31 | Asp Gln Trp Glu Lys Phe Gly Leu Glu Lys |
| 41 | Arg Gln Glu Ser Leu Glu Leu Ile Arg Lys |
| 51 | Gly Tyr Thr Gln Gln Leu Ala Phe Arg Gln |
| 61 | Lys Ser Ser Ala Tyr Ala Ala Phe Gln Tyr |
| 71 | Arg Pro Pro Ser Thr Trp Leu Thr Ala Tyr |
| 81 | Val Val Lys Val Phe Ala Leu Ala Ala Asn |
| 91 | Leu Ile Ala Ile Asp Ser Lys Asp Leu Cys |
| 101 | Glu Thr Val Lys Trp Leu Ile Leu Glu Lys |
| 111 | Gln Lys Pro Asp Gly Ile Phe Gln Glu Asp |
| 121 | Gly Pro Val Ile His Gln Glu Met Ile Gly |
| 131 | Gly Phe Arg Asp Thr Arg Glu Lys Asp Val |
| 141 | Ser Leu Thr Ala Phe Val Leu Ile Ala Leu |
| 151 | His Glu Ala Lys Asp Ile Cys Glu Ala Gln |
| 161 | Val Asn Ser Leu Gly Arg Ser Ile Ala Lys |
| 171 | Ala Gly Asp Phe Leu Glu Asn His Tyr Arg |
| 181 | Glu Leu Arg Arg Pro Tyr Thr Val Ala Ile |
| 191 | Ala Ala Tyr Ala Leu Ala Leu Leu Gly Lys |
| 201 | Leu Glu Gly Asp Arg Leu Thr Lys Phe Leu |
| 211 | Asn Thr Ala Lys Glu Lys Asn Arg Trp Glu |
| 221 | Glu Pro Asn Gln Lys Leu Tyr Asn Val Glu |
| 231 | Ala Thr Ser Tyr Ala Leu Leu Ala Leu Leu |
| 241 | Ala Arg Lys Asp Tyr Asp Thr Thr Pro Pro |
| 251 | Val Val Arg Trp Leu Asn Glu Gln Arg Tyr |
| 261 | Tyr Gly Gly Gly Tyr Gly Ser Thr Gln Ala |
| 271 | Thr Phe Met Val Phe Gln Ala Leu Ala Gln |
| 281 | Tyr Gln Lys Asp Val Pro Asp His Lys Glu |
| 291 | Leu Asn Leu Asp Val Ser Ile Gln Leu Pro |

SEQID10: Nucleotide sequence of cow C3d

| | | |
|---|---|---|
| 1 | CACCTTATCC AAACCCCCTC CGGCTGTGGG GAGCAGAACA TGATTGGTAT | 50 |
| 51 | GACGCCCACG GTCATCGCCG TGCACTACCT GGACAGCACC GACCAGTGGG | 100 |
| 101 | AGAAGTTCGG CTTGGAGAAG CGGCAGGAGT CCCTGGAGCT CATCAGAAAG | 150 |
| 151 | GGGTACACCC AGCAGCTGGC CTTCAGACAA AAAAGCTCAG CCTACGCCGC | 200 |
| 201 | CTTCCAATAT CGGCCCCCCA GCACCTGGCT GACAGCCTAC GTGGTCAAGG | 250 |
| 251 | TCTTTGCACT GGCCGCCAAC CTCATCGCCA TAGACTCCAA GGACCTCTGT | 300 |
| 301 | GAGACCGTCA AATGGCTGAT CCTGGAGAAG CAGAAGCCTG ATGGAATCTT | 350 |
| 351 | CCAGGAGGAT GGGCCTGTGA TACACCAAGA AATGATTGGT GGCTTCAGGG | 400 |
| 401 | ACACCAGGGA GAAAGATGTG TCCCTTACAG CCTTTGTTCT CATCGCGCTG | 450 |
| 451 | CACGAGGCTA AAGACATTTG CGAGGCACAG GTCAACAGCC TGGGCCGCAG | 500 |
| 501 | CATCGCTAAG GCAGGAGACT TCCTCGAAAA CCACTACAGA GAGTTGCGAA | 550 |
| 551 | GACCATATAC TGTGGCCATT GCTGCCTATG CCCTGGCTTT GTTGGGCAAG | 600 |
| 601 | CTGGAGGGTG ACCGCCTCAC CAAATTTCTG AACACAGCCA AAGAAAAGAA | 650 |
| 651 | CCGCTGGGAG GAACCCAACC AGAAGCTCTA CAATGTGGAG GCCACGTCCT | 700 |
| 701 | ACGCCCTCTT GGCTCTGCTG GCACGCAAAG ACTACGACAC TACGCCTCCT | 750 |
| 751 | GTCGTGCGCT GGCTCAACGA GCAGAGATAC TATGGAGGTG GTTATGGCTC | 800 |
| 801 | CACGCAGGCC ACTTTCATGG TGTTCCAAGC CTTGGCCCAA TACCAGAAGG | 850 |
| 851 | ATGTTCCTGA TCACAAGGAG CTGAACCTGG ATGTGTCCAT CCAACTGCCC | 900 |

SEQID11: Amino acid sequence of C3d from horse

| | |
|---|---|
| 1 | His Leu Ile Val Thr Pro Ser Gly Cys Gly |
| 11 | Glu Gln Asn Met Ile Ser Met Thr Pro Thr |
| 21 | Val Ile Ala Val His Tyr Leu Asp Gln Thr |
| 31 | Glu Gln Trp Glu Lys Phe Gly Leu Glu Lys |
| 41 | Arg Gln Glu Ser Leu Glu Leu Ile Lys Lys |
| 51 | Gly Tyr Thr Gln Gln Leu Ala Tyr Arg Gln |
| 61 | Pro Ser Ser Ala Tyr Ala Ala Phe Leu Ser |
| 71 | Arg Pro Pro Ser Thr Trp Leu Thr Ala Tyr |
| 81 | Val Val Lys Val Phe Ala Leu Ala Ser Asn |
| 91 | Leu Ile Ala Ile Asp Ser Gln Val Leu Cys |
| 101 | Gly Ala Val Lys Trp Leu Ile Leu Gln Lys |
| 111 | Gln Lys Pro Asp Gly Val Phe Gln Glu Asp |
| 121 | Gly Pro Val Ile His Gln Glu Met Ile Gly |
| 131 | Gly Phe Arg Asn Ala Glu Glu Lys Asp Val |
| 141 | Ser Leu Thr Ala Phe Val Leu Ile Ala Leu |
| 151 | Gln Glu Ala Lys Asp Ile Cys Glu Gly Gln |
| 161 | Val Asn Ser Leu Ala Arg Ser Ile Ile Lys |
| 171 | Ala Gly Asp Phe Leu Glu Ala His Tyr Asn |
| 181 | Asn Leu Arg Arg Pro Tyr Ser Val Ala Ile |
| 191 | Ala Gly Tyr Ala Leu Ala Gln Met Gly Lys |
| 201 | Leu Glu Asp Pro Leu Leu Asn Lys Phe Leu |
| 211 | Ser Ala Ala Thr Asp Arg Asn Arg Trp Glu |
| 221 | Glu Pro Gly Gln Lys Leu Tyr Asn Val Glu |
| 231 | Ala Thr Ser Tyr Ala Leu Leu Ala Leu Leu |
| 241 | Leu Leu Arg Asp Phe Asp Ser Val Pro Pro |
| 251 | Val Val Arg Trp Leu Asn Glu Gln Arg Tyr |
| 261 | Tyr Gly Gly Gly Tyr Gly Ser Thr Gln Ala |
| 271 | Thr Phe Met Val Phe Gln Ala Leu Ala Gln |
| 281 | Tyr Gln Lys Asp Val Pro Asn His Lys Asp |
| 291 | Leu Asn Leu Asp Val Ser Ile Asn Leu Pro |

SEQID12: Nucleotide sequence of horse C3d

| | | |
|---|---|---|
| 1 | CACCTCATCG TGACGCCCTC GGGCTGCGGC GAGCAGAACA TGATTAGCAT | 50 |
| 51 | GACGCCCACG GTCATCGCAG TGCATTACCT GGACCAGACC GAGCAGTGGG | 100 |
| 101 | AGAAGTTCGG CCTGGAGAAG CGGCAGGAGT CCTTGGAGCT CATCAAGAAG | 150 |
| 151 | GGGTACACCC AGCAGCTGGC CTACAGACAA CCCAGCTCAG CCTATGCAGC | 200 |
| 201 | CTTCCTGAGC CGGCCGCCCA GCACCTGGCT GACAGCCTAC GTGGTCAAGG | 250 |
| 251 | TCTTCGCTCT GGCCTCCAAC CTCATCGCCA TCGACTCCCA GGTCCTCTGT | 300 |
| 301 | GGGGCTGTCA AATGGCTGAT CCTCCAGAAG CAGAAGCCAG ATGGAGTCTT | 350 |
| 351 | CCAGGAGGAC GGGCCCGTGA TACATCAAGA AATGATTGGT GGCTTCCGGA | 400 |
| 401 | ATGCGGAGGA GAAAGACGTG TCCCTCACAG CCTTTGTTCT CATCGCACTG | 450 |
| 451 | CAGGAAGCTA AAGATATTTG CGAGGGACAG GTCAACAGCC TGGCACGCAG | 500 |
| 501 | CATCATTAAG GCAGGAGACT TCCTTGAAGC CCACTATAAT AACCTGCGGA | 550 |
| 551 | GACCATATTC TGTGGCCATT GCTGGCTACG CCCTGGCCCA GATGGGCAAG | 600 |
| 601 | CTGGAGGACC CCCTCCTCAA CAAATTCCTG AGCGCAGCCA CAGACAGGAA | 650 |
| 651 | CCGCTGGGAG GAGCCTGGCC AGAAGCTCTA CAATGTAGAG GCCACATCCT | 700 |
| 701 | ACGCCCTCTT GGCCCTGCTG CTGCTCAGAG ACTTTGACTC TGTGCCTCCG | 750 |
| 751 | GTGGTGCGCT GGCTCAACGA ACAGAGATAC TACGGAGGTG GCTATGGCTC | 800 |
| 801 | CACCCAGGCC ACCTTCATGG TGTTCCAAGC CTTGGCTCAG TACCAAAAGG | 850 |
| 851 | ATGTCCCTAA CCACAAGGAC CTGAACCTCG ATGTTTCCAT CAACCTGCCC | 900 |

SEQID13: Amino acid sequence of C3d from marmoset

| | |
|---|---|
| 1 | His Leu Ile Val Thr Pro Ser Gly Cys Gly |
| 11 | Glu Gln Asn Met Ile Gly Met Thr Pro Thr |
| 21 | Val Ile Ala Val His Tyr Leu Asp Gln Thr |
| 31 | Glu Gln Trp Glu Lys Phe Gly Leu Glu Lys |
| 41 | Arg Gln Gly Ala Val Glu Leu Ile Lys Lys |
| 51 | Gly Tyr Ala Gln Gln Leu Ala Phe Lys Gln |
| 61 | Pro Ser Ser Ala Tyr Ala Ala Phe Leu Asn |
| 71 | Arg Pro Pro Ser Thr Trp Leu Thr Ala Tyr |
| 81 | Val Val Lys Val Phe Ser Leu Ala Val Asn |
| 91 | Leu Ile Ala Ile Asp Ser Gln Val Leu Cys |
| 101 | Gly Ala Val Lys Trp Leu Ile Leu Glu Lys |
| 111 | Gln Lys Pro Asp Gly Val Phe Gln Glu Asp |
| 121 | Gly Pro Val Ile His Gln Glu Met Ile Gly |
| 131 | Gly Phe Arg Asn Thr Gln Glu Lys Asp Met |
| 142 | Ala Leu Thr Ala Phe Val Leu Ile Ser Leu |
| 151 | Gln Glu Ala Lys Asp Ile Cys Glu Glu Leu |
| 161 | Val Asn Ser Leu Pro Arg Ser Ile Ile Asp |
| 171 | Ala Gly Asn Phe Leu Glu Ala Asn Tyr Met |
| 181 | Asn Leu Gln Arg Ser Tyr Thr Val Ala Ile |
| 191 | Ala Gly Tyr Ala Leu Ala Gln Leu Asp Lys |
| 201 | Leu Asn Gly Pro Leu Leu Asn Lys Phe Leu |
| 211 | Ser Thr Ala Lys Asp Lys Asn Arg Trp Glu |
| 221 | Glu Pro Gly Gln Gln Leu Tyr Asn Val Glu |
| 231 | Ala Thr Ser Tyr Ala Leu Leu Ala Leu Leu |
| 241 | Gln Met Lys Asp Phe Asp Phe Val Pro Pro |
| 251 | Val Val Arg Trp Leu Asn Glu Gln Arg Tyr |
| 261 | Tyr Gly Gly Gly Tyr Gly Ser Thr Gln Ala |
| 271 | Thr Phe Met Val Phe Gln Ala Leu Ala Gln |
| 281 | Tyr Gln Lys Asp Val Pro Asp His Lys Glu |
| 291 | Leu Asn Leu Asp Val Ser Leu Gln Leu Pro |

SEQID14: Nucleotide sequence of marmoset C3d

| | | |
|---|---|---|
| 1 | CACCTCATCG TGACCCCCTC GGGCTGTGGG GAACAGAACA TGATTGGCAT | 50 |
| 51 | GACACCCACG GTCATCGCGG TGCATTACCT GGATCAAACG GAGCAGTGGG | 100 |
| 101 | AGAAGTTCGG CTTGGAGAAG CGGCAGGGAG CCGTGGAGCT CATCAAGAAG | 150 |
| 151 | GGGTACGCAC AGCAGCTGGC CTTCAAACAA CCCAGCTCTG CCTATGCGGC | 200 |
| 201 | CTTCCTGAAC CGGCCACCCA GCACCTGGCT GACCGCCTAC GTGGTCAAGG | 250 |
| 251 | TCTTCTCTCT GGCCGTCAAC CTCATTGCCA TTGACTCCCA AGTCCTCTGT | 300 |
| 301 | GGGGCTGTTA AATGGCTGAT CCTGGAGAAG CAGAAGCCTG ATGGGGTCTT | 350 |
| 351 | CCAGGAGGAT GGGCCCGTGA TACACCAAGA AATGATTGGT GGCTTCCGGA | 400 |
| 401 | ACACCCAGGA GAAAGACATG GCCCTCACGG CCTTTGTTCT CATCTCGCTG | 450 |
| 451 | CAGGAGGCTA AAGATATTTG CGAGGAGCTT GTCAACAGCC TGCCACGCAG | 500 |
| 501 | CATCATTGAT GCAGGAAACT TCCTTGAAGC CAACTACATG AACCTACAGA | 550 |
| 551 | GATCCTACAC TGTGGCCATC GCTGGCTATG CCCTGGCCCA GCTGGACAAA | 600 |
| 601 | CTGAACGGGC CTCTTCTCAA CAAATTTCTG AGCACAGCCA AAGATAAGAA | 650 |
| 651 | CCGCTGGGAG GAGCCTGGTC AGCAGCTCTA CAACGTGGAG GCCACATCCT | 700 |
| 701 | ATGCCCTCTT GGCCCTCCTG CAGATGAAAG ACTTCGACTT TGTGCCTCCC | 750 |
| 751 | GTCGTGCGTT GGCTCAATGA GCAGAGATAC TACGGTGGCG GCTATGGCTC | 800 |
| 801 | TACCCAGGCC ACCTTCATGG TGTTCCAAGC CTTGGCCCAA TACCAAAAGG | 850 |
| 851 | ACGTCCCTGA CCACAAGGAA CTGAACCTGG ATGTGTCCCT CCAACTGCCA | 900 |

SEQID 15: Amino acid sequence of C3d from pig

| | |
|---|---|
| 1 | His Leu Ile Gln Thr Pro Ser Gly Cys Gly |
| 11 | Glu Gln Asn Met Ile Gly Met Thr Pro Thr |
| 21 | Val Ile Ala Val His Tyr Leu Asp Ser Thr |
| 31 | Glu Gln Trp Glu Lys Phe Gly Leu Glu Lys |
| 41 | Arg Gln Glu Ala Leu Glu Leu Ile Lys Lys |
| 51 | Gly Tyr Thr Gln Gln Leu Ala Phe Arg Gln |
| 61 | Lys Asn Ser Ala Phe Ala Ala Phe Gln Asp |
| 71 | Arg Leu Ser Ser Thr Trp Leu Thr Ala Tyr |
| 81 | Val Val Lys Val Phe Ala Met Ala Ala Asn |
| 91 | Leu Ile Ala Ile Asp Ser Gln Val Leu Cys |
| 101 | Gly Ala Val Lys Trp Leu Ile Leu Glu Lys |
| 111 | Gln Lys Pro Asp Gly Val Phe Glu Glu Asn |
| 121 | Gly Pro Val Ile His Gln Glu Met Ile Gly |
| 131 | Gly Phe Lys Asn Thr Glu Glu Lys Asp Val |
| 141 | Ser Leu Thr Ala Phe Val Leu Ile Ala Leu |
| 151 | Gln Glu Ala Lys Asp Ile Cys Glu Pro Gln |
| 161 | Val Asn Ser Leu Leu Arg Ser Ile Asn Lys |
| 171 | Ala Arg Asp Phe Leu Ala Asp Tyr Tyr Leu |
| 181 | Glu Leu Lys Arg Pro Tyr Thr Val Ala Ile |
| 191 | Ala Gly Tyr Ala Leu Ala Leu Ser Asp Lys |
| 201 | Leu Asp Glu Pro Phe Leu Asn Lys Leu Leu |
| 211 | Ser Thr Ala Lys Glu Arg Asn Arg Trp Glu |
| 221 | Glu Pro Gly Gln Lys Leu Tyr Asn Val Glu |
| 231 | Ala Thr Ser Tyr Ala Leu Leu Ala Leu Leu |
| 241 | Val Val Lys Asp Phe Asp Ser Val Pro Pro |
| 251 | Ile Val Arg Trp Leu Asn Glu Gln Arg Tyr |
| 261 | Tyr Gly Gly Gly Tyr Gly Ser Thr Gln Ala |
| 271 | Thr Phe Met Val Phe Gln Ala Leu Ala Gln |
| 281 | Tyr Gln Lys Asp Val Pro Asp His Lys Asp |
| 291 | Leu Asn Leu Asp Val Ser Ile His Leu Pro |

SEQID16: Nucleotide sequence of pig C3d

| | | |
|---|---|---|
| 1 | CACCTCATCC AAACCCCCTC CGGCTGTGGG GAGCAGAACA TGATCGGCAT | 50 |
| 51 | GACGCCCACA GTCATCGCTG TGCACTACCT GGACAGCACC GAACAATGGG | 100 |
| 101 | AGAAGTTCGG CCTGGAGAAG AGGCAGGAAG CCTTGGAGCT CATCAAGAAG | 150 |
| 151 | GGGTACACCC AGCAACTGGC CTTCAGACAA AAGAACTCAG CCTTTGCCGC | 200 |
| 201 | CTTCCAGGAC CGGCTGTCCA GCACCTGGCT GACAGCCTAT GTGGTCAAGG | 250 |
| 251 | TCTTCGCTAT GGCAGCCAAC CTCATCGCCA TCGACTCCCA GGTCCTCTGT | 300 |
| 301 | GGGGCCGTCA AATGGCTGAT CCTGGAGAAG CAGAAGCCTG ATGGAGTCTT | 350 |
| 351 | CGAGGAGAAT GGGCCCGTGA TACACCAAGA AATGATTGGT GGCTTCAAGA | 400 |
| 401 | ACACTGAGGA GAAAGACGTG TCCCTGACAG CCTTTGTTCT CATCGCGCTG | 450 |
| 451 | CAGGAGGCTA AAGACATCTG TGAACCACAG GTCAATAGCC TGTTGCGCAG | 500 |
| 501 | CATCAATAAG GCAAGAGACT TCCTCGCAGA CTACTACCTA GAATTAAAAA | 550 |
| 551 | GACCATATAC TGTGGCCATT GCTGGTTATG CCCTGGCTCT ATCTGACAAG | 600 |
| 601 | CTGGATGAGC CCTTCCTCAA CAAACTTCTG AGCACAGCCA AAGAAAGGAA | 650 |
| 651 | CCGCTGGGAG GAACCTGGCC AGAAGCTCTA CAATGTGGAG GCCACATCCT | 700 |
| 701 | ACGCCCTCTT GGCTCTGCTG GTAGTCAAAG ACTTTGACTC TGTCCCTCCT | 750 |
| 751 | ATTGTGCGCT GGCTCAATGA GCAGAGATAC TACGGAGGTG GCTACGGATC | 800 |
| 801 | TACCCAGGCC ACTTTCATGG TGTTCCAAGC CTTGGCCCAA TACCAGAAGG | 850 |
| 851 | ATGTCCCTGA TCACAAGGAT CTGAACCTGG ATGTGTCCAT CCACCTGCCC | 900 |

SEQID 17: Amino acid sequence of C3d from dog

| | |
|---|---|
| 1 | His Leu Ile Val Thr Pro Ser Gly Cys Gly |
| 11 | Gln Glu Asn Met Ile Gly Met Thr Pro Thr |
| 21 | Val Ile Ala Leu His Tyr Leu Asp Glu Thr |
| 31 | Gln Glu Trp Asp Lys Phe Gly Leu Gln Lys |
| 41 | Arg Glu Gln Ala Leu Gln Leu Ile Lys Lys |
| 51 | Gly Tyr Thr Glu Glu Leu Ala Phe Arg Glu |
| 61 | Pro Asn Ser Ala Phe Ala Ala Phe Glu Asn |
| 71 | Arg Pro Ser Ser Thr Trp Leu Thr Ala Tyr |
| 81 | Val Val Lys Val Phe Ser Leu Ala Thr Asn |
| 91 | Leu Ile Ala Ile Gln Ala Glu Val Leu Cys |
| 101 | Gly Ala Val Lys Trp Leu Ile Leu Gln Lys |
| 111 | Glu Lys Pro Asp Gly Ile Phe Glu Gln Asp |
| 121 | Gly Pro Val Ile His Gln Glu Met Thr Gly |
| 131 | Gly Phe Arg Glu Ala Glu Glu Lys Ser Val |
| 141 | Ser Leu Thr Ala Phe Val Leu Ile Ala Leu |
| 151 | Lys Glu Ala Glu Asp Ile Cys Ile Gly Gln |
| 161 | Val Asn Ile Leu Pro Ser Ser Ile Glu Lys |
| 171 | Ala Gly Asn Tyr Leu Ala Ala His Tyr Gln |
| 181 | Asn Leu Arg Arg Pro Tyr Ser Val Ala Ile |
| 191 | Ala Gly Tyr Ala Leu Ala His Leu Asp Lys |
| 201 | Leu Glu Gly Asp Asn Leu Arg Lys Phe Leu |
| 211 | Asn Thr Ala Arg Asp Arg Asn Arg Trp Val |
| 221 | Glu Pro Gly Lys Lys Leu Tyr Asn Val Glu |
| 231 | Ala Thr Ser Tyr Ala Leu Leu Ala Leu Leu |
| 241 | Leu Leu Lys Asp Phe Asp Asn Val Pro Pro |
| 251 | Val Val Arg Trp Leu Asn Gln Glu Arg Tyr |
| 261 | Tyr Gly Gly Gly Tyr Gly Ser Thr Glu Ala |
| 271 | Thr Phe Met Val Phe Glu Ala Leu Ala Glu |
| 281 | Tyr Glu Lys Asp Val Pro Asn His Lys Asp |
| 291 | Leu Asn Leu Glu Val Ser Ile Asn Leu Pro |
| 301 | Ser Arg Ser Thr Gln Ile Lys His His Ile |
| 311 | Val Trp |

SEQID 18: Nucleotide sequence of dog C3d

| | | |
|---|---|---|
| 1 | CACCTCATCG TGACCCCGTC GGGCTGCGGG GAGCAGAACA TGATCGGCAT | 50 |
| 51 | GACGCCCACC GTCATCGCCC TGCATTACCT GGACCAAACC GAGCAGTGGG | 100 |
| 101 | ACAAGTTCGG GCTGGAGAAG CGCCAGGAGG CCTTGGAGCT CATCAAGAAG | 150 |
| 151 | GGATACACCC AACAGCTGGC CTTCAGACAA CCCAACTCGG CCTTCGCTGC | 200 |
| 201 | CTTCCAGAAC CGGCCATCCA GCACCTGGCT GACAGCCTAC GTGGTCAAGG | 250 |
| 251 | TCTTCTCTCT GGCCACCAAC CTCATCGCCA TTGAAGCCCA GGTTCTCTGC | 300 |
| 301 | GGGGCTGTCA AATGGCTGAT CCTGGAGAAG CAGAAGCCCG ATGGGATCTT | 350 |
| 351 | CCAGGAGGAT GGGCCTGTGA TCCACCAAGA GATGACCGGT GGCTTCCGGG | 400 |
| 401 | AAGCTGAGGA GAAGTCTGTG TCCCTCACGG CCTTTGTTCT CATAGCACTA | 450 |
| 451 | AAAGAGGCTG AAGATATTTG TATAGGACAG GTTAACATCT TGCCAAGCAG | 500 |
| 501 | CATCGAAAAG GCAGGAAATT ATCTTGCAGC CCACTACCAG AACCTGAGGA | 550 |
| 551 | GACCATATTC TGTGGCCATT GCTGGCTACG CCCTGGCCCA TCTAGACAAA | 600 |
| 601 | CTGGAGGGAG ATAACCTCAG AAAATTTCTG AACACAGCCA GAGACAGGAA | 650 |
| 651 | CCGCTGGGTG GAGCCTGGCA AGAAGCTCTA CAATGTGGAA GCCACATCCT | 700 |
| 701 | ACGCCCTCTT GGCCCTGCTG CTGCTCAAAG ACTTTGACAA TGTACCTCCT | 750 |
| 751 | GTCGTCCGCT GGCTCAATGA GCAGAGATAC TACGGAGGTG GCTATGGCTC | 800 |
| 801 | CACCCAGGCC ACCTTCATGG TGTTCCAAGC CTTGGCCCAA TACCAGAAGG | 850 |
| 851 | ATGTCCCCAA CCACAAGGAC CTGAACCTGC AAGTGTCCAT CAACCTGCCC | 900 |
| 901 | AGCCGCAGCA CAGAGATCAA GCACCACATT GTCTGG 936 | |

SEQID19: Nucleotide sequence of first variant cow C3d

| | | |
|---|---|---|
| 1 | CACCTGATTC AAACTCCTAG CGGGTGCGGC GAGCAAAACA TGATTGGGAT | 50 |
| 51 | GACCCCTACA GTTATCGCAG TCCACTACCT CGATTCCACT GACCAATGGG | 100 |
| 101 | AAAAATTCGG ACTGGAAAAA CGCCAAGAGA GCCTCGAGTT GATTAGGAAA | 150 |
| 151 | GGCTACACTC AGCAACTCGC ATTCCGTCAG AAATCTTCCG CTTACGCGGC | 200 |
| 201 | TTTCCAGTAT AGGCCTCCTT CCACATGGCT CACTGCATAC GTCGTGAAAG | 250 |
| 251 | TGTTTGCCTT GGCTGCTAAC TTGATCGCTA TTGACAGCAA AGACCTGTGT | 300 |
| 301 | GAAACGGTGA AGTGGCTCAT TCTCGAAAAG CAAAAGCCAG ATGGTATTTT | 350 |
| 351 | TCAAGAGGAC GGCCCAGTCA TTCACCAGGA AATGATCGGC GGTTTTCGCG | 400 |
| 401 | ACACTAGAGA GAAGGATGTC AGTCTCACCG CTTTTGTGCT GATTGCCCTC | 450 |
| 451 | CACGAAGCAA AAGATATCTG CGAAGCTCAG GTGAATTCTC TCGGGCGTAG | 500 |
| 501 | TATCGCCAAG GCTGGTGATT TCTTGGAGAA CCACTACCGT GAGCTCCGCA | 550 |
| 551 | GGCCATATAC CGTCGCTATT GCCGCTTATG CACTCGCCCT CCTGGGGAAG | 600 |
| 601 | TTAGAGGGAG ATCGTCTGAC TAAATTCCTC AACACGGCAA AAGAGAAGAA | 650 |
| 651 | TCGCTGGGAA GAACCTAACC AAAAGTTGTA TAATGTTGAG GCAACCAGCT | 700 |
| 701 | ACGCACTGCT GGCACTCCTC GCTAGGAAGG ACTATGACAC CACTCCTCCA | 750 |
| 751 | GTGGTCCGTT GGTTGAACGA ACAGCGCTAC TACGGTGGCG GCTATGGGTC | 800 |
| 801 | TACCCAGGCT ACATTTATGG TCTTCCAGGC TCTGGCTCAA TATCAGAAAG | 850 |
| 851 | ATGTCCCAGA TCACAAGGAA CTCAATCTCG ATGTTTCTAT CCAATTGCCT | 900 |

SEQID 20: Nucleotide sequence of second variant cow C3d

| | | |
|---|---|---|
| 1 | CACTTGATCC AGACACCATC TGGTTGTGGA GAACAAAATA TGATCGGCAT | 50 |
| 51 | GACACCAACC GTGATTGCTG TTCACTATTT GGATAGTACA GATCAGTGGG | 100 |
| 101 | AAAAGTTTGG GCTCGAGAAA AGACAGGAAT CTCTTGAACT GATCCGCAAA | 150 |
| 151 | GGATATACAC AACAGTTGGC TTTTCGCCAA AAGTCCAGCG CATATGCAGC | 200 |
| 201 | ATTTCAATAC CGCCCACCAT CTACTTGGTT GACCGCTTAT GTTGTTAAGG | 250 |
| 251 | TTTTCGCTCT CGCAGCAAAT CTGATTGCAA TCGATTCTAA GGATTTGTGC | 300 |
| 301 | GAGACTGTTA AATGGTTAAT CTTGGAGAAA CAGAAACCTG ACGGGATCTT | 350 |
| 351 | TCAGGAAGAT GGTCCTGTTA TCCACCAGGA GATGATCGGG GGATTTAGAG | 400 |
| 401 | ATACACGTGA AAAAGACGTT TCTCTGACTG CATTCGTCTT GATCGCTTTG | 450 |
| 451 | CACGAGGCCA AGGACATCTG TGAGGCCCAA GTTAATAGTT TGGGTAGATC | 500 |
| 501 | CATTGCAAAA GCCGGGGACT TTCTGGAAAA TCACTATAGG GAACTGAGAC | 550 |
| 551 | GCCCTTACAC AGTAGCAATC GCAGCATACG CTTTGGCACT GCTCGGAAAA | 600 |
| 601 | CTCGAAGGGG ACAGATTAAC AAAGTTTTTG AATACCGCTA AGGAAAAAAA | 650 |
| 651 | CAGATGGGAA GAGCCAAATC AGAAACTGTA CAACGTAGAA GCTACTAGTT | 700 |
| 701 | ATGCTTTGCT CGCCTTGTTG GCCAGAAAAG ATTACGATAC AACCCCACCT | 750 |
| 751 | GTAGTAAGAT GGCTGAATGA GCAAAGGTAT TACGGGGGAG GATACGGAAG | 800 |
| 801 | CACTCAAGCA ACCTTCATGG TTTTTCAAGC ACTCGCACAG TACCAAAAGG | 850 |
| 851 | ACGTTCCTGA CCACAAAGAG TTGAACTTGG ACGTCAGCAT TCAGCTCCCA | 900 |

SEQID21: Nucleotide sequence of first variant pig C3d

| | | |
|---|---|---|
| 1 | CACCTGATTC AAACGCCATC AGGGTGCGGA GAGCAAAACA TGATTGGTAT | 50 |
| 51 | GACCCCAACG GTGATCGCAG TCCACTATCT CGATTCAACG GAACAGTGGG | 100 |
| 101 | AGAAATTCGG ATTAGAGAAA AGACAAGAAG CGCTCGAATT GATCAAAAAG | 150 |
| 151 | GGCTATACGC AGCAGTTAGC TTTTAGACAG AAGAATTCCG CGTTCGCTGC | 200 |
| 201 | GTTCCAAGAC AGACTTAGTT CAACATGGTT AACTGCGTAT GTTGTGAAAG | 250 |
| 251 | TTTTCGCCAT GGCTGCGAAT CTGATTGCGA TCGATTCACA AGTGTTATGT | 300 |
| 301 | GGCGCTGTGA AATGGTTAAT TCTTGAAAAG CAAAAGCCAG ATGGCGTGTT | 350 |
| 351 | TGAGGAAAAT GGCCCAGTCA TTCACCAGGA AATGATCGGC GGATTTAAGA | 400 |
| 401 | ATACGGAGGA AAAAGATGTA TCATTAACGG CATTTGTCTT AATTGCACTT | 450 |
| 451 | CAAGAGGCGA AGGACATTTG TGAGCCTCAG GTGAACAGTT TATTAAGAAG | 500 |
| 501 | TATTAACAAG GCGCGTGACT TTCTGGCGGA TTACTATCTC GAGTTGAAGA | 550 |
| 551 | GGCCTTATAC GGTAGCTATC GCGGGATACG CACTCGCCTT GTCAGATAAG | 600 |
| 601 | TTAGATGAAC CATTTCTGAA TAAATTACTT TCAACGGCAA AAGAGAGAAA | 650 |
| 651 | TCGTTGGGAA GAACCTGGAC AAAAGTTATA TAACGTAGAG GCAACGAGTT | 700 |
| 701 | ACGCACTTCT TGCCCTCTTA GTGGTTAAGG ACTTCGACAG CGTACCTCCA | 750 |
| 751 | ATTGTCAGAT GGCTGAACGA GCAACGCTAC TATGGGGGAG GGTACGGCAG | 800 |
| 801 | CACGCAGGCA ACGTTTATGG TCTTCCAGGC GTTAGCGCAG TACCAAAAGG | 850 |
| 851 | ACGTACCAGA TCACAAAGAT TTAAACCTCG ACGTCAGTAT CCACTTACCA | 900 |

SEQID22: Nucleotide sequence of second variant pig C3d

| | | |
|---|---|---|
| 1 | CACTTAATCC AGACTCCTAG TGGATGTGGC GAACAGATTA TGATCGGGAT | 50 |
| 51 | GACACCTACC GTAATTGCGG TTCACTACTT AGACAGTACA GAGCAATGGG | 100 |
| 101 | AAAAGTTTGG GCTCGAAAAG CGCCAGGAGG CTCTTGAGTT AATTAAGAAA | 150 |
| 151 | GGTTACACAC AACAACTCGC ATTCAGGCAA AAAAACAGTG CATTTGCGGC | 200 |
| 201 | ATTTCAGGAT CGCTTAAGCA GTACGTGGCT CACCGCATAC GTCGTAAAGG | 250 |
| 251 | TGTTTGCGAT GGCCGCAAAC TTAATCGCAA TTGACAGTCA GGTACTGTGC | 300 |
| 301 | GGAGCGGTTA AGTGGCTTAT CTTAGAGAAA CAGAAACCTG ACGGGGTATT | 350 |
| 351 | CGAAGAGAAC GGTCCTGTTA TCCACCAAGA GATGATTGGG GGTTTCAAAA | 400 |
| 401 | ACACCGAAGA GAAGGACGTT AGTCTCACCG CTTTCGTGCT GATCGCCTTA | 450 |
| 451 | CAGGAAGCCA AAGATATCTG CGAACCACAA GTAAATTCAC TCCTTCGTTC | 500 |
| 501 | AATCAATAAA GCCAGGGATT TCTTAGCCGA CTATTACTTG GAACTCAAAC | 550 |
| 551 | GTCCATACAC CGTTGCGATT GCCGGGTATG CTTTAGCGCT CAGCGACAAA | 600 |
| 601 | CTCGACGAGC CTTTCTTAAA CAAGCTGTTA AGTACCGCTA AGGAACGCAA | 650 |
| 651 | CAGGTGGGAG GAGCCAGGTC AGAAACTTTA CAATGTTGAA GCTACCTCAT | 700 |
| 701 | ATGCTTTATT AGCGTTACTC GTCGTAAAAG ATTTTGATTC AGTGCCACCT | 750 |
| 751 | ATCGTACGTT GGTTAAATGA ACAGAGGTAT TACGGTGGCG GATATGGGTC | 800 |
| 801 | AACACAAGCG ACCTTCATGG TATTTCAAGC ACTCGCACAA TATCAGAAAG | 850 |
| 851 | ATGTGCCTGA CCACAAGGAC CTGAATTTAG ATGTATCAAT TCACCTTCCT | 900 |

SEQID23: Nucleotide sequence of first variant dog C3d

| | | |
|---|---|---|
| 1 | CACTTAATCG TCACTCCAAG TGGATGCGGC GAACAGAATA TGATCGGAAT | 50 |
| 51 | GACACCAACG GTAATTGCGC TCCACTATTT AGACCAGACT GAGCAATGGG | 100 |
| 101 | ATAAGTTTGG ACTCGAAAAG AGGCAGGAAG CGCTCGAGTT AATCAAAAAG | 150 |
| 151 | GGCTATACGC AGCAGTTAGC GTTTAGGCAG CCAAATTCCG CGTTTGCAGC | 200 |
| 201 | GTTCCAAAAC AGACCATCAT CAACGTGGCT TACGGCTTAT GTCGTTAAAG | 250 |
| 251 | TGTTCTCATT AGCGACTAAT CTTATTGCAA TCGAGGCTCA GGTCCTTTGT | 300 |
| 301 | GGCGCGGTTA AATGGTTAAT TCTCGAAAAG CAAAAACCAG ACGGCATTTT | 350 |
| 351 | CCAAGAGGAC GGCCCTGTAA TTCACCAAGA AATGACGGGC GGTTTTCGCG | 400 |
| 401 | AAGCGGAAGA GAAATCAGTA AGTCTTACCG CGTTCGTGCT GATTGCGTTG | 450 |
| 451 | AAAGAAGCCG AAGACPTTTG CATTGGGCAG GTCAATATCC TCCCTAGTTC | 500 |
| 501 | AATTGAAAAA GCTGGCAACT ATCTGGCCGC ACACTATCAG AATCTCCGTA | 550 |
| 551 | GGCCTTATAG CGTCGCAATA GCCGGTTACG CATTAGGACA CCTCGATAAA | 600 |
| 601 | TTAGAGGGTG ACAACTTACG CAAGTTTCTC AATACGGCAA GGGATAGAAA | 650 |
| 651 | TCGTTGGGTA GAACCAGGGA AGAAACTTTA TAATGTAGAG GCGACAAGTT | 700 |
| 701 | ACGCGCTGCT CGCTCTTTTA TTACTAAAGG ACTTCGACAA CGTCCCACCT | 750 |
| 751 | GTGGTGAGGT GGCTTAATGA ACAGCGTTAC TATGGCGGAG GATACGGATC | 800 |
| 801 | AACGCAAGCT ACATTCATGG TCTTTCAAGC GCTCGCGCAA TATCAGAAAG | 850 |
| 851 | ATGTGCCAAA TCACAAGGAT CTCAATTTAC AAGTAAGTAT CAATCTCCCA | 900 |

SEQID24: Nucleotide sequence of second variant dog C3d

| | | |
|---|---|---|
| 1 | CACCTGATTG TAACGCCTAG CGGTTGTGGA GAGCAAAACA TGATTGGGAT | 50 |
| 51 | GACCCCTACT GTGATCGCAT TACACTACCT CGATCAAACA GAACAGTGGG | 100 |
| 101 | ACAAATTCGG TTTAGAGAAA CGTCAAGAGG CTTTAGAACT GATTAAGAAA | 150 |
| 151 | GGTTACACCC AACAACTCGC TTTCCGTCAA CCTAACAGTG CTTTCGCGGC | 200 |
| 201 | TTTTCAGAAT CGTCCTAGTA GTACATGGTT AACCGCATAC GTAGTGAAGG | 250 |
| 251 | TATTTAGTCT TGCAACGAAC TTAATCGCTA TCGAAGCGCA AGTGTTGTGC | 300 |
| 301 | GGAGCCGTGA AGTGGCTCAT CTTAGAGAAA CAGAAGCCTG ATGGTATCTT | 350 |
| 351 | TCAGGAAGAT GGACCAGTCA TCCACCAGGA GATGACTGGA GGGTTCAGAG | 400 |
| 401 | AGGCCGAGGA AAAGAGCGTG TCATTGACAG CTTTTGTCTT AATCGCCCTC | 450 |
| 451 | AAGGAGGCGG AGGATATCTG TATCGGCCAA GTAAACATTT TACCATCATC | 500 |
| 501 | TATCGAGAAG GCCGGTAATT ACTTAGCTGC GCACTACCAA AACTTAAGAC | 550 |
| 551 | GCCCATACTC AGTTGCGATT GCAGGGTATG CGCTCGCGCA CTTGGACAAG | 600 |
| 601 | CTCGAAGGCG ATAATCTGAG GAAATTCTTA AACACTGCCC GTGACCGCAA | 650 |
| 651 | CAGATGGGTC GAGCCAGGAA AAAAGTTGTA CAACGTCGAA GCTACCTCAT | 700 |
| 701 | ATGCATTACT TGCCTTACTC CTTTTGAAAG ATTTTGATAA TGTACCTCCA | 750 |
| 751 | GTAGTTCGTT GGTTGAACGA GCAAAGGTAT TACGGGGGCG GTTATGGTAG | 800 |
| 801 | TACTCAGGCG ACGTTTATGG TATTCCAGGC ATTAGCACAG TACCAAAAGG | 850 |
| 851 | ACGTACCTAA CCACAAAGAC TTAAACCTTC AGGTCTCAAT TAACTTACCT | 900 |

SEQID 25 Rhesus macaque (Macaca mulatta) C3d

ACCCCCTCGGGCTGCGGAGAACAGAACATGATCACCATGACGCCCACAGT
CATCGCTGTGCATTACCTGGATGAAACGGAACAGTGGGAGAAGTTCGGCC
CGGAGAAGCGGCAGGGGGCCTTGGAGCTCATCAAGAAGGGGTACACCCAG
CAGCTGGCCTTCAGACAACCCAGCTCTGCCTTTGCGGCCTTCCTGAACCG
GGCACCCAGCACCTGGCTGACCGCCTACGTGGTCAAGGTCTTCTCTCTGG
CTGTCAACCTCATTGCCATCGACTCCCAGGTCCTCTGCGGGGCTGTTAAA
TGGCTGATCCTGGAGAAGCAGAAGCCCGACGGGGTCTTCCAGGAGGATGC
GCCCGTGATACATCAAGAAATGACTGGTGGATTCCGGAACACCAACGAGA
AAGACATGGCCCTCACGGCCTTTGTTCTCATCTCGCTGCAAGAGGCTAAA
GAGATTTGCGAGGAGCAGGTCAACAGCCTGCCCGGCAGCATCACTAAAGC
AGGAGACTTCCTTGAAGCCAACTACATGAACCTACAGAGATCCTACACTG
TGGCCATCGCTGCCTATGCCCTGGCCCAGATGGGCAGGCTGAAGGGACCT
CTTCTCAACAAATTTCTGACCACAGCCAAAGATAAGAACCGCTGGGAGGA
GCCTGGTCAGCAGCTCTACAATGTGGAGGCCACATCCTATGCCCTCTTGG
CCCTACTGCAGCTAAAAGACTTTGACTTTGTGCCTCCCGTCGTGCGTTGG
CTCAATGAACAGAGATACTACGGTGGTGGCTATGGCTCTACCCAGGCCAC
CTTCATGGTGTTCCAAGCCTTGGCTCAATACCAAAGGATGTCCCTGATCA
CAAGGAACTGAACCTGGATGTGTCCCTCCAACTGCCC

SEQID 26 First variant Rhesus C3d

ACGCCAAGCGGATCAGGCGAGCAGAATATGATCACTATGACACCAACCGT
AATTGCGGTCCATTATCTCGACGAAACCGAACAGTGGGAAAAATTTGGGC
CGGAAAAGCGTCAAGGCGCTCTCGAGTTGATCAAGAAAGGCTACACGCAA
CAGTTAGCGTTCCGTCAACCATCATCAGCGTTCGCCGCTTTCCTGAATCG
TGCGCCATCAACGTGGCTCACAGCGTATGTAGTGAAGGTATTTAGCCTCG
CCGTAAATTTAATCGCGATTGACAGTCAAGTGTTATGCGGCGCGGTCAAG
TGGCTCATTCTTGAAAAGCAAAAGCCAGATGGCGTATTCCAAGAGGACGC
CCCAGTCATCCACCAAGAGATGACAGGCGGCTTTCGCAATACTAATGAGA
AGGACATGGCGTTAACCGCGTTTGTCTTAATCAGTTTACAGGAAGCCAAA
GAAATTTGTGAGGAACAGGTAAATAGTTTACCTGGGAGTATTACGAAAGC
GGGCGATTTCTTAGAAGCAAATTACATGAATCTCCAACGCTCATACACGG
TAGCGATCGCGGCTTATGCCTTAGCGCAGATGGGGAGATTAAAAGGCCCA
TTACTGAACAAGTTTTTAACAACCGCAAAAGACAAGAATAGGTGGGAGGA
ACCAGGCCAACAACTTTATAACGTCGAAGCAACGTCATACGCATTATTAG
CACTCTTACAACTCAAGGACTTCGACTTCGTACCACCTGTGGTACGGTGG
CTTAACGAACAAAGGTATTACGGGGGCGGATACGGCAGCACGCAAGCGAC
TTTCATGGTCTTTCAAGCACTCGCACAGTACCAGAAGGATGTTCCTGATC
ACAAGGAATTAAACTTAGATGTCAGTCTGCAGTTACCA

SEQID 27 Second variant Rhesus C3d

ACTCCTTCAGGGAGTGGAGAACAAAACATGATTACAATGACCCCTACAGT
GATCGCCGTACACTACTTAGATGAGACAGAGCAATGGGAGAAATTCGGTC
CCGAGAAAAGACAGGGAGCGTTAGAACTTATTAAAAAGGGATATACACAG
CAACTCGCTTTTAGGCAGCCTAGTAGCGCATTTGCTGCGTTTCTCAACAG
AGCCCCTAGTACATGGTTAACGGCTTACGTCGTAAAAGTGTTCTCATTAG
CGGTGAACCTGATTGCAATCGATTCGCAGGTACTGTGTGGAGCCGTGAAA
TGGTTAATCTTAGAGAAACAGAAACCTGACGGAGTGTTTCAGGAAGATGC
ACCTGTAATTCACCAGGAAATGACCGGGGGCTTCAGAAACACAAACGAAA
AAGATATGGCTCTGACAGCTTTCGTGCTGATTTCCCTCCAAGAGGCGAAG
GAGATCTGCGAAGAGCAAGTGAACTCACTCCCAGGATCAATCACCAAGGC
CGGGGACTTTCTGGAGGCGAACTATATGAACTTGCAGAGGAGCTATACCG
TCGCAATTGCCGCATACGCGCTCGCACAAATGGGACGTCTCAAAGGACCT
CTGTTAAATAAATTCCTCACGACGGCGAAGGATAAAAACCGATGGGAAGA
ACCTGGGCAACAGTTGTACAATGTAGAGGCGACCAGTTATGCGCTGCTCG
CGTTGCTCCAGTTGAAAGATTTTGATTTTGTCCCTCCAGTAGTCAGATGG
TTGAATGAGCAGCGTTACTATGGAGGCGGGTATGGATCAACACAGGCAAC
GTTTATGGTATTCCAGGCGTTAGCGCAATATCAAAAAGACGTGCCAGACC
ACAAAGAGCTTAATCTCGACGTATCATTACAACTCCCT

SEQID 28 Third variant Rhesus C3d

ACACCGTCTGGTAGCGGTGAGCAAAATATGATAACCATGACTCCGACTGT
TATAGCAGTTCACTATTTAGACGAGACTGAACAATGGGAAAAGTTTGGAC
CGGAAAAAAGGCAAGGTGCACTGGAATTAATAAAAAAAGGTTATACGCAG
CAACTAGCGTTCAGGCAGCCGTCCAGCGCTTTCGCAGCATTTCTGAACAG
GGCTCCGTCCACTTGGTTGACGGCATATGTCGTGAAAGTTTTTAGTTTGG
CAGTTAACTTGATAGCGATCGATAGCCAGGTTTTGTGTGGTGCAGTAAAG
TGGTTGATACTCGAAAAGCAAAAGCCGGATGGTGTTTTTCAAGAAGACGC
CCCGGTTATCCATCAGGAGATGACTGGAGGTTTCAGGAATACCAATGAAA
AGGATATGGCATTGACTGCATTCGTATTGATAAGCTTGCAAGAAGCAAAG
GAGATATGTGAAGAACAAGTTAATTCCTTGCCGGGTTCCATAACAAAGGC
TGGTGATTTTCTCGAGGCTAATTATATGAATCTGCAACGAAGTTATACAG
TTGCTATAGCAGCCTACGCACTCGCTCAAATGGGTCGCTTGAAGGGTCCG
CTCCTGAATAAGTTCTTGACTACTGCTAAGGACAAAAATAGATGGGAAGA
GCCGGGACAGCAACTGTATAATGTTGAAGCTACTAGCTACGCTTTGCTGG
CTCTGTTGCAACTGAAGGATTTCGATTTCGTTCCCCCGGTTGTTAGGTGG
TTAAACGAGCAACGCTATTATGGCGGAGGTTACGGGTCGACTCAAGCTAC
ATTTATGGTTTTTCAGGCTCTGGCCCAGTATCAGAAAGATGTCCCCGATC
ATAAGGAGCTCAATCTGGACGTTAGCTTGCAGTTGCCG

SEQID 29 Rhesus C3d amino acid sequence (single letter code)

TPSGSGEQNMITMTPTVIAVHYLDETEQWEKFGPEKRQGALELIKKGYTQ
QLAFRQPSSAFAAFLNRAPSTWLTAYVVKVFSLAVNLIAIDSQVLCGAVK
WLILEKQKPDGVFQEDAPVIHQEMTGGFRNTNEKDMALTAFVLISLQEAK
EICEEQVNSLPGSITKAGDFLEANYMNLQRSYTVAIAAYALAQMGRLKGP
LLNKFLTTAKDKNRWEEPGQQLYNVEATSYALLALLQLKDFDFVPPVVRW
LNEQRYYGGGYGSTQATFMVFQALAQYQKDVPDHKELNLDVSLQLP

SEQID 30 Rhesus C3d amino acid sequence (three letter code)

ThrProSerGlySerGlyGluGlnAsnMetIleThrMetThrProThr
ValIleAlaVaLHisTyrLeuAspGluThrGluGlnTrpGluLysPhe
GlyProGluLysArgGlnGlyAlaLeuGluLeuIleLysLysGlyTyr
ThrGlnGlnLeuAlaPheArgGlnProSerSerAlaPheAlaAlaPhe
LeuAsnArgAlaProSerThrTrpLeuThrAlaTyrValValLysVal
PheSerLeuAlaValAsnLeuIleAlaIleAspSerGlnValLeuCys
GlyAlaValLysTrpLeuIleLeuGluLysGlnLysproAspGlyVal
PheGlnGluAspAlaProValIleHisGlnGluMetThrGlyGlyPhe
ArgAsnThrAsnGluLysAspMetAlaLeuThrAlaPheValLeuIle
SerLeuGlnGluAlaLysGluIleCysGluGluGlnValAsnSerLeu
ProGlySerIleThrLysAlaGlyAspPheLeuGluAlaAsnTyrMet
AsnLeuGlnArgSerTyrThrValAlaIleAlaAlaTyrAlaLeuAla
GlnMetGlyArgLeuLysGlyProLeuLeuAsnLysPheLeuThrThr
AlaLysAspLysAsnArgTrpGluGluProGlyGlnGlnLeuTyrAsn
ValGluAlaThrSerTyrAlaLeuLeuAlaLeuLeuGlnLeuLysAsp
PheAspPheValProProValValArgTrpLeuAsnGluGlnArgTyr
TyrGlyGlyGlyTyrGlySerThrGlnAlaThrPheMetValPheGln
AlaLeuAlaGlnTyrGlnLysAspValProAspHisLysGluLeuAsn
LeuAspValSerLeuGlnLeuPro

1. A C3d polypeptide in substantially isolated form comprising or consisting of an amino acid sequence corresponding to SEQ ID NO: 9, 11, 13, 15, 17, 29, or 30, or a derivative thereof which retains immunostimulatory activity.

2. A recombinant C3d polypeptide comprising or consisting of an amino acid sequence corresponding to SEQ ID NO: 9, 11, 13, 15, 17, 29, or 30, or a derivative thereof which retains immunostimulatory activity.

3. A nucleic acid in substantially isolated form which encodes a C3d polypeptide according to claim 1 or 2.

4. A nucleic acid according to claim 3 comprising or consisting of a nucleic acid sequence corresponding to SEQ ID NO: 10, 12, 14, 16, 18, or 25.

5. A variant nucleic acid sequence for use in a veterinary vaccine encoding a naturally occurring non human protein or polypeptide, or a fragment thereof (preferably at least 15 amino acid residues long) which retains a desired activity or property, which by virtue of third base redundancy and/or other variations permissible within an amino acid codon, is non-identical to the naturally occurring DNA sequence encoding that protein, polypeptide, or fragment.

6. A variant nucleic acid sequence according to claim 5 in which the protein or polypeptide is an immunostimulatory protein or polypeptide, or a fragment thereof which retains immunostimulatory activity, such as a complement protein, polypeptide or fragment, preferably a C3d polypeptide or fragment.

7. A variant nucleic acid sequence according to claim 6 which encodes a C3d polypeptide comprising or consisting of an amino acid sequence corresponding to SEQ ID NO: 9, 11, 13, 15, 17, 29, or 30, or a fragment thereof which retains immunostimulatory activity.

8. A variant nucleic acid sequence according to claim 7 comprising or consisting of a DNA sequence corresponding to SEQ ID NO: 19, 20, 21, 22, 23, 24, 26, 27, or 28.

9. A linear concatamer comprising at least two non identical nucleic acid sequences which by virtue of third base redundancy and/or each other variations permissible within an amino acid codon each encode the same naturally occurring non human protein or polypeptide, or a fragment thereof which retains a desired activity or property.

10. A linear concatamer according to claim 9 in which not more than one of the non identical nucleic acid sequences is a naturally occurring nucleic acid sequence.

11. A linear concatamer according to claim 9 or 10 in which the naturally occurring protein, polypeptide, or fragment is species specific.

12. A linear concatamer according to claim 11 in which the species specific polypeptide is a C3d polypeptide comprising or consisting of an amino acid sequence corresponding to SEQ ID NO: 9, 11, 13, 15, 17, 29, or 30, or a fragment thereof which retains immunostimulatory activity.

13. A construct comprising a variant nucleic acid sequence according to any of claims 5 to 8, or a linear concatamer according to claim 9 fused in frame to one or more nucleic acid sequences encoding an antigen.

14. A DNA immunisation vector comprising a construct according to claim 13.

15. A veterinary composition comprising a variant nucleic acid sequence according to any of claims 5 to 8, a linear concatamer according to claim 9, a construct according to claim 13, or a DNA immunisation vector according to claim 14, together with a physiologically acceptable excipient, carrier, or diluent.

16. Use of a variant nucleic acid sequence according to any of claims 5 to 8, a linear concatamer according to any of claims 9 to 12, a construct according to claim 13, a DNA immunisation vector according to claim 14, or a veterinary composition according to claim 15, for inducing an immune response to an antigen in a non human animal.

17. Use of a variant nucleic acid sequence according to any of claims 5 to 8, a linear concatamer according to any of claims 9 to 12, a construct according to claim 13, a DNA immunisation vector according to claim 14, or a veterinary composition according to claim 15, for the manufacture of a medicament for inducing an immune response to an antigen in a non human animal.

18. A method of inducing an immune response to an antigen in a non human animal which comprises administering a variant nucleic acid sequence according to any of claims 5 to 8, a linear concatamer according to any of claims 9 to 12, a construct according to claim 13, a DNA immunisation vector according to claim 14, or a veterinary composition according to claim 15, to the animal.

19. A construct comprising a linear concatamer according to claim 11 or 12 fused to one or more sequences encoding an antigen.

20. A DNA immunisation vector comprising a construct according to claim 19.

21. A veterinary composition comprising a linear concatamer according to claim 11 or 12, a construct according to claim 19, or a DNA immunisation vector according to claim 20, together with a physiologically acceptable excipient, carrier, or diluent.

22. Use of a linear concatamer according to claim 11 or 12, a construct according to claim 19, a DNA immunisation vector according to claim 20, or a veterinary composition according to claim 21, for inducing an immune response to an antigen in an animal of the same species as the encoded species specific protein, polypeptide, or fragment.

23. Use of a linear concatamer according to claim 11 or 12, a construct according to claim 19, a DNA immunisation vector according to claim 20, or a veterinary composition according to claim 21, for the manufacture of a medicament for inducing an immune response to an antigen in an animal of the same species as the encoded species specific protein, polypeptide, or fragment.

24. A method of inducing an immune response to an antigen in an animal which comprises administering a linear concatamer according to claim 11 or 12, a construct according to claim 19, a DNA immunisation vector according to claim 20, or a veterinary composition according to claim 21, to an animal of the same species as the encoded species specific protein, polypeptide, or fragment.

25. A vector comprising a nucleic acid according to claim 3 or 4, a variant nucleic acid sequence according to any of claims 5 to 8, a linear concatamer according to any of claims 9 to 12, or a construct according to claim 13.

26. An expression vector encoding a C3d polypeptide or derivative according to claim 1 which is capable of directing expression of the C3d polypeptide or derivative in a prokaryotic or eukaryotic expression system.

27. An expression vector according to claim 25 comprising a nucleic acid according to claim 3 or 4.

28. An expression vector comprising a variant nucleic acid sequence according to any of claims 5 to 8 which is capable of directing expression of the naturally occurring protein or polypeptide, or a fragment thereof, encoded by the variant DNA sequence in a prokaryotic or eukaryotic expression system.

29. An expression vector comprising a linear concatamer according to any of claims 9 to 12 which is capable of directing expression of the protein, polypeptide or fragment encoded by the linear concatamer in a prokaryotic or eukaryotic expression system.

30. An expression vector comprising a construct according to claim 13 which is capable of directing expression of the protein, polypeptide or fragment and the antigen encoded by the construct in a prokaryotic or eukaryotic expression system.

31. An expression vector according to any of claims 26 to 30 which is a replicable expression vector.

32. Use of an expression vector according to any claims 26 to 31 with a eukaryotic or prokaryotic expression system.

33. A host cell comprising a vector according to claim 25, or an expression vector according to any of claims 26 to 31.

34. A host cell comprising an expression vector according to any of claims 36 to 31 which is capable of expressing the protein, polypeptide or fragment and, if present, the antigen encoded by the expression vector.

35. A method of high level production of a polypeptide or protein, or a fragment thereof, which comprises:
culturing a host cell comprising an expression vector according to claim 29 under conditions for expression of the polypeptide, protein or fragment encoded by the expression vector in the host cell; and
recovering the polypeptide, protein or fragment expressed by the host cell.

36. A method according to claim 35 in which expression vector is a replicable expression vector.

37. A method according to claim 35 or 36 which further comprises transforming the host cell with the expression vector.

38. A method according to claim 37 which further comprises preparing the expression vector.

39. A method according to any of claims 35 to 38 in which the linear concatamer of the expression vector is fused in frame to one or more nucleic acid sequences encoding an antigen and the antigen is expressed by the host cell, and is recovered from the host cell, with the polypeptide, protein or fragment.

40. A method of producing a C3d polypeptide, or derivative, of claim 1 which comprises:
culturing a host cell comprising an expression vector according to claim 25 under conditions for expression of the polypeptide or derivative in the host cell; and
recovering the polypeptide or derivative expressed by the host cell.

41. A method of producing a protein, polypeptide, or fragment encoded by a variant DNA sequence according to any of claims 5 to 8 which comprises:
culturing a host cell comprising an expression vector according to claim 28 under conditions for expression of the protein, polypeptide or derivative in the host cell; and
recovering the protein, polypeptide or derivative expressed by the host cell.

42. A method according to claim 41 in which the variant nucleic acid of the expression vector is fused in frame to one or more nucleic acid sequences encoding an antigen and the antigen is expressed by the host cell, and is recovered from the host cell, with the polypeptide, protein or fragment.

43. A method according to any of claims 40 to 42 in which the expression vector is a replicable expression vector.

44. A method according to any of claims 40 to 43 which further comprises transforming the host cell with the expression vector.

45. A method according to claim 44 which further comprises preparing the expression vector.

46. An oligomeric protein comprising at least two, preferably at least three, identical C3d polypeptides, each polypeptide comprising or consisting of a sequence corresponding to SEQ ID NO: 9, 11, 13, 15, 17, 29, or 30, or a fragment thereof which retain immunostimulatory activity.

47. An oligomeric protein according to claim 46 fused to an antigen.

48. A recombinant live organism comprising an oligomeric protein according to claim 46 or 47.

49. A recombinant live organism comprising a variant nucleic acid sequence according to any of claims 5 to 8, a linear concatamer according to any of claims 9 to 12, or a construct according to claim 13, operably linked to sequences capable of directing expression of the amino acid sequence encoded by the variant nucleic acid, linear concatamer, or construct in the recombinant live organism.

50. A recombinant live organism according to claim 48 or 49 which is an attenuated recombinant live organism.

51. A veterinary composition comprising a fusion protein according to claim 46 or 47, or a recombinant live organism according to any of claims 48 to 50, together with a physiologically acceptable carrier, excipient or diluent.

52. Use of an oligomeric protein according to claim 46 or 47, a recombinant live organism according to any of claims 48 to 50, or a veterinary composition according to claim 51, for inducing an immune response to an antigen in an animal of the same species as the species from which the C3d polypeptide or fragment is derived.

53. Use of an oligomeric protein according to claim 46 or 47, a recombinant live organism according to any of claims 48 to 50, or a veterinary composition according to claim 51, for the manufacture of a medicament for inducing an immune response to an antigen in an animal of the same species as the species from which the C3d polypeptide or fragment is derived.

54. A method of inducing an immune response to an antigen in an animal which comprises administering an oligomeric protein according to claim 46 or 47, a recombinant live organism according to any of claims 48 to 50, or a veterinary composition according to claim 51, to an animal of the same species as the species from which the C3d polypeptide or fragment is derived.

55. An oligonucleotide in substantially isolated form comprising or consisting of a sequence corresponding to any of SEQ ID NOS: 1-8.

56. Use of an oligonucleotide according to claim 55 to clone nucleic acid encoding a C3d polypeptide.

57. A method of cloning nucleic acid encoding a C3d polypeptide or fragment thereof, which comprises:
i) obtaining a nucleic acid sample from a vertebrate, the sample including nucleic acid encoding the C3d polypeptide or fragment;
ii) amplifying nucleic acid of the nucleic acid sample by PCR using a nucleic acid primer comprising or consisting of a sequence corresponding to any of SEQ ID NOS. 1, 3, 5 or 7 together with a primer comprising or consisting of a sequence corresponding to any of SEQ ID NOS. 2, 4, 6 or 8 to; and
iii) obtaining the amplified nucleic acid.

58. A method according to claim 57 which further comprises amplifying the nucleic acid amplified in step (ii) by nested PCR using nucleic acid primers capable of priming to internal sequence of the amplified nucleic acid.

59. A method according to claim 58 in which the internal nucleic acid primers are selected from nucleic acid primers comprising or consisting of a sequence corresponding to SEQ ID NOS: 1, 2, 3, 6, 7 and 8.

60. A method according to any of claims 57 to 59 in which the nucleic acid sample is derived from a tissue sample, preferably a liver sample, of the vertebrate.

61. A method according to any of claims 57 to 60 in which the nucleic acid sample comprises an RNA sample, preferably a total RNA preparation, and RNA of the RNA sample is reverse transcribed using a nucleic acid primer comprising or consisting of a sequence corresponding to SEQ ID NO. 2, 4, 6, or 8 to provide DNA which is then amplified according to step (ii).

62. A non human C3d polypeptide in substantially isolated form or a derivative thereof which retains immunostimulatory activity, excluding mouse, rat, guinea pig, rabbit, sheep, chicken, cobra, lamprey, toad, carp, trout, and sea urchin C3d.

63. A nucleic acid in substantially isolated form encoding a C3d polypeptide or derivative according to claim 62.

64. A nucleic acid in substantially isolated form comprising or consisting of a sequence corresponding to any of SEQ ID NOS: 10, 12, 14, 16, 18-28.

65. A method of preparing a linear concatamer which comprises condensing together at least two non identical nucleic acid sequences which by virtue of third base redundancy and/or other variations permissible within an amino acid codon each encode the same naturally occurring non human protein or polypeptide, or a fragment thereof which retains a desired activity or property.


SEQUENCE LISTING (30 sequences)

SEQ ID NO: 1 - DNA, 27 bp, Artificial Sequence
  PCR primer FARM 1
tgyggrgarcagaacatgatyggcatg

SEQ ID NO: 2 - DNA, 26 bp, Artificial Sequence
  PCR primer FARM 2
ccgtagtatctyasntcrttgagcca

SEQ ID NO: 3 - DNA, 23 bp, Artificial Sequence
  PCR primer FARM 3
ggagtcttcgaggagaatgggcc

SEQ ID NO: 4 - DNA, 28 bp, Artificial Sequence
  PCR primer FARM 4
gtgtgtcwggrrcraagccrgtcatcat

SEQ ID NO: 5 - DNA, 27 bp, Artificial Sequence
  PCR primer FARM 5
gtratgcaggacttcttcatygacctg

SEQ ID NO: 6 - DNA, 24 bp, Artificial Sequence
  PCR primer FARM 6
ggctgtcagggacacgtctttctc

SEQ ID NO: 7 - DNA, 25 bp, Artificial Sequence
  PCR primer FARM 7
gcaagggaccccmgtggcccagatg

SEQ ID NO: 8 - DNA, 23 bp, Artificial Sequence
  PCR primer FARM 8
gycaccaccgacaakgtgccttg

SEQ ID NO: 9 - protein, 300 aa, Bos sp.
HLIQTPSGCGEQNMIGMTPTVIAVHYLDSTDQWEKFGLEKRQESLELIRKGYTQQLAFRQKSSAYAAFQYRPPSTWLTAYVVKVFALAANLIAIDSKDLCETVKWLILEKQKPDGIFQEDGPVIHQEMIGGFRDTREKDVSLTAFVLIALHEAKDICEAQVNSLGRSIAKAGDFLENHYRELRRPYTVAIAAYALALLGKLEGDRLTKFLNTAKEKNRWEEPNQKLYNVEATSYALLALLARKDYDTTPPVVRWLNEQRYYGGGYGSTQATFMVFQALAQYQKDVPDHKELNLDVSIQLP

SEQ ID NO: 10 - DNA, 900 bp, Bos sp.
  CDS (1)..(900)
caccttatccaaaccccctccggctgtggggagcagaacatgattggtatgacgcccacggtcatcgccgtgcactacctggacagcaccgaccagtgggagaagttcggcttggagaagcggcaggagtccctggagctcatcagaaaggggtacacccagcagctggccttcagacaaaaaagctcagcctacgccgccttccaatatcggccccccagcacctggctgacagcctacgtggtcaaggtctttgcactggccgccaacctcatcgccatagactccaaggacctctgtgagaccgtcaaatggctgatcctggagaagcagaagcctgatggaatcttccaggaggatgggcctgtgatacaccaagaaatgattggtggcttcagggacaccagggagaaagatgtgtcccttacagcctttgttctcatcgcgctgcacgaggctaaagacatttgcgaggcacaggtcaacagcctgggccgcagcatcgctaaggcaggagacttcctcgaaaaccactacagagagttgcgaagaccatatactgtggccattgctgcctatgccctggctttgttgggcaagctggagggtgaccgcctcaccaaatttctgaacacagccaaagaaaagaaccgctgggaggaacccaaccagaagctctacaatgtggaggccacgtcctacgccctcttggctctgctggcacgcaaagactacgacactacgcctcctgtcgtgcgctggctcaacgagcagagatactatggaggtggttatggctccacgcaggccactttcatggtgttccaagccttggcccaataccagaaggatgttcctgatcacaaggagctgaacctggatgtgtccatccaactgccc

SEQ ID NO: 11 - protein, 300 aa, Equus caballus
HLIVTPSGCGEQNMISMTPTVIAVHYLDQTEQWEKFGLEKRQESLELIKKGYTQQLAYRQPSSAYAAFLSRPPSTWLTAYVVKVFALASNLIAIDSQVLCGAVKWLILQKQKPDGVFQEDGPVIHQEMIGGFRNAEEKDVSLTAFVLIALQEAKDICEGQVNSLARSIIKAGDFLEAHYNNLRRPYSVAIAGYALAQMGKLEDPLLNKFLSAATDRNRWEEPGQKLYNVEATSYALLALLLLRDFDSVPPVVRWLNEQRYYGGGYGSTQATFMVFQALAQYQKDVPNHKDLNLDVSINLP

SEQ ID NO: 12 - DNA, 900 bp, Equus caballus
  CDS (1)..(900)
cacctcatcgtgacgccctcgggctgcggcgagcagaacatgattagcatgacgcccacggtcatcgcagtgcattacctggaccagaccgagcagtgggagaagttcggcctggagaagcggcaggagtccttggagctcatcaagaaggggtacacccagcagctggcctacagacaacccagctcagcctatgcagccttcctgagccggccgcccagcacctggctgacagcctacgtggtcaaggtcttcgctctggcctccaacctcatcgccatcgactcccaggtcctctgtggggctgtcaaatggctgatcctccagaagcagaagccagatggagtcttccaggaggacgggcccgtgatacatcaagaaatgattggtggcttccggaatgcggaggagaaagacgtgtccctcacagcctttgttctcatcgcactgcaggaagctaaagatatttgcgagggacaggtcaacagcctggcacgcagcatcattaaggcaggagacttccttgaagcccactataataacctgcggagaccatattctgtggccattgctggctacgccctggcccagatgggcaagctggaggaccccctcctcaacaaattcctgagcgcagccacagacaggaaccgctgggaggagcctggccagaagctctacaatgtagaggccacatcctacgccctcttggccctgctgctgctcagagactttgactctgtgcctccggtggtgcgctggctcaacgaacagagatactacggaggtggctatggctccacccaggccaccttcatggtgttccaagccttggctcagtaccaaaaggatgtccctaaccacaaggacctgaacctcgatgtttccatcaacctgccc

SEQ ID NO: 13 - protein, 300 aa, Callithrix sp.
HLIVTPSGCGEQNMIGMTPTVIAVHYLDQTEQWEKFGLEKRQGAVELIKKGYAQQLAFKQPSSAYAAFLNRPPSTWLTAYVVKVFSLAVNLIAIDSQVLCGAVKWLILEKQKPDGVFQEDGPVIHQEMIGGFRNTQEKDMALTAFVLISLQEAKDICEELVNSLPRSIIDAGNFLEANYMNLQRSYTVAIAGYALAQLDKLNGPLLNKFLSTAKDKNRWEEPGQQLYNVEATSYALLALLQMKDFDFVPPVVRWLNEQRYYGGGYGSTQATFMVFQALAQYQKDVPDHKELNLDVSLQLP

SEQ ID NO: 14 - DNA, 900 bp, Callithrix sp.
  CDS (1)..(900)
cacctcatcgtgaccccctcgggctgtggggaacagaacatgattggcatgacacccacggtcatcgcggtgcattacctggatcaaacggagcagtgggagaagttcggcttggagaagcggcagggagccgtggagctcatcaagaaggggtacgcacagcagctggccttcaaacaacccagctctgcctatgcggccttcctgaaccggccacccagcacctggctgaccgcctacgtggtcaaggtcttctctctggccgtcaacctcattgccattgactcccaagtcctctgtggggctgttaaatggctgatcctggagaagcagaagcctgatggggtcttccaggaggatgggcccgtgatacaccaagaaatgattggtggcttccggaacacccaggagaaagacatggccctcacggcctttgttctcatctcgctgcaggaggctaaagatatttgcgaggagcttgtcaacagcctgccacgcagcatcattgatgcaggaaacttccttgaagccaactacatgaacctacagagatcctacactgtggccatcgctggctatgccctggcccagctggacaaactgaacgggcctcttctcaacaaatttctgagcacagccaaagataagaaccgctgggaggagcctggtcagcagctctacaacgtggaggccacatcctatgccctcttggccctcctgcagatgaaagacttcgactttgtgcctcccgtcgtgcgttggctcaatgagcagagatactacggtggcggctatggctctacccaggccaccttcatggtgttccaagccttggcccaataccaaaaggacgtccctgaccacaaggaactgaacctggatgtgtccctccaactgcca

SEQ ID NO: 15 - protein, 300 aa, Sus sp.
HLIQTPSGCGEQNMIGMTPTVIAVHYLDSTEQWEKFGLEKRQEALELIKKGYTQQLAFRQKNSAFAAFQDRLSSTWLTAYVVKVFAMAANLIAIDSQVLCGAVKWLILEKQKPDGVFEENGPVIHQEMIGGFKNTEEKDVSLTAFVLIALQEAKDICEPQVNSLLRSINKARDFLADYYLELKRPYTVAIAGYALALSDKLDEPFLNKLLSTAKERNRWEEPGQKLYNVEATSYALLALLVVKDFDSVPPIVRWLNEQRYYGGGYGSTQATFMVFQALAQYQKDVPDHKDLNLDVSIHLP

SEQ ID NO: 16 - DNA, 900 bp, Sus sp.
  CDS (1)..(900)
cacctcatccaaaccccctccggctgtggggagcagaacatgatcggcatgacgcccacagtcatcgctgtgcactacctggacagcaccgaacaatgggagaagttcggcctggagaagaggcaggaagccttggagctcatcaagaaggggtacacccagcaactggccttcagacaaaagaactcagcctttgccgccttccaggaccggctgtccagcacctggctgacagcctatgtggtcaaggtcttcgctatggcagccaacctcatcgccatcgactcccaggtcctctgtggggccgtcaaatggctgatcctggagaagcagaagcctgatggagtcttcgaggagaatgggcccgtgatacaccaagaaatgattggtggcttcaagaacactgaggagaaagacgtgtccctgacagcctttgttctcatcgcgctgcaggaggctaaagacatctgtgaaccacaggtcaatagcctgttgcgcagcatcaataaggcaagagacttcctcgcagactactacctagaattaaaaagaccatatactgtggccattgctggttatgccctggctctatctgacaagctggatgagcccttcctcaacaaacttctgagcacagccaaagaaaggaaccgctgggaggaacctggccagaagctctacaatgtggaggccacatcctacgccctcttggctctgctggtagtcaaagactttgactctgtccctcctattgtgcgctggctcaatgagcagagatactacggaggtggctacggatctacccaggccactttcatggtgttccaagccttggcccaataccagaaggatgtccctgatcacaaggatctgaacctggatgtgtccatccacctgccc

SEQ ID NO: 17 - protein, 312 aa, Canis familiaris
HLIVTPSGCGEQNMIGMTPTVIALHYLDQTEQWDKFGLEKRQEALELIKKGYTQQLAFRQPNSAFAAFQNRPSSTWLTAYVVKVFSLATNLIAIEAQVLCGAVKWLILEKQKPDGIFQEDGPVIHQEMTGGFREAEEKSVSLTAFVLIALKEAEDICIGQVNILPSSIEKAGNYLAAHYQNLRRPYSVAIAGYALAHLDKLEGDNLRKFLNTARDRNRWVEPGKKLYNVEATSYALLALLLLKDFDNVPPVVRWLNEQRYYGGGYGSTQATFMVFQALAQYQKDVPNHKDLNLQVSINLPSRSTEIKHHIVW

SEQ ID NO: 18 - DNA, 936 bp, Canis familiaris
  CDS (1)..(936)
cacctcatcgtgaccccgtcgggctgcggggagcagaacatgatcggcatgacgcccaccgtcatcgccctgcattacctggaccaaaccgagcagtgggacaagttcgggctggagaagcgccaggaggccttggagctcatcaagaagggatacacccaacagctggccttcagacaacccaactcggccttcgctgccttccagaaccggccatccagcacctggctgacagcctacgtggtcaaggtcttctctctggccaccaacctcatcgccattgaagcccaggttctctgcggggctgtcaaatggctgatcctggagaagcagaagcccgatgggatcttccaggaggatgggcctgtgatccaccaagagatgaccggtggcttccgggaagctgaggagaagtctgtgtccctcacggcctttgttctcatagcactaaaagaggctgaagatatttgtataggacaggttaacatcttgccaagcagcatcgaaaaggcaggaaattatcttgcagcccactaccagaacctgaggagaccatattctgtggccattgctggctacgccctggcccatctagacaaactggagggagataacctcagaaaatttctgaacacagccagagacaggaaccgctgggtggagcctggcaagaagctctacaatgtggaagccacatcctacgccctcttggccctgctgctgctcaaagactttgacaatgtacctcctgtcgtccgctggctcaatgagcagagatactacggaggtggctatggctccacccaggccaccttcatggtgttccaagccttggcccaataccagaaggatgtccccaaccacaaggacctgaacctgcaagtgtccatcaacctgcccagccgcagcacagagatcaagcaccacattgtctgg

SEQ ID NO: 19 - DNA, 900 bp, Artificial Sequence
  Nucleotide sequence of first variant Bos sp. C3d
cacctgattcaaactcctagcgggtgcggcgagcaaaacatgattgggatgacccctacagttatcgcagtccactacctcgattccactgaccaatgggaaaaattcggactggaaaaacgccaagagagcctcgagttgattaggaaaggctacactcagcaactcgcattccgtcagaaatcttccgcttacgcggctttccagtataggcctccttccacatggctcactgcatacgtcgtgaaagtgtttgccttggctgctaacttgatcgctattgacagcaaagacctgtgtgaaacggtgaagtggctcattctcgaaaagcaaaagccagatggtatttttcaagaggacggcccagtcattcaccaggaaatgatcggcggttttcgcgacactagagagaaggatgtcagtctcaccgcttttgtgctgattgccctccacgaagcaaaagatatctgcgaagctcaggtgaattctctcgggcgtagtatcgccaaggctggtgatttcttggagaaccactaccgtgagctccgcaggccatataccgtcgctattgccgcttatgcactcgccctcctggggaagttagagggagatcgtctgactaaattcctcaacacggcaaaagagaagaatcgctgggaagaacctaaccaaaagttgtataatgttgaggcaaccagctacgcactgctggcactcctcgctaggaaggactatgacaccactcctccagtggtccgttggttgaacgaacagcgctactacggtggcggctatgggtctacccaggctacatttatggtcttccaggctctggctcaatatcagaaagatgtcccagatcacaaggaactcaatctcgatgtttctatccaattgcct

SEQ ID NO: 20 - DNA, 900 bp, Artificial Sequence
  Nucleotide sequence of second variant Bos sp. C3d
cacttgatccagacaccatctggttgtggagaacaaaatatgatcggcatgacaccaaccgtgattgctgttcactatttggatagtacagatcagtgggaaaagtttgggctcgagaaaagacaggaatctcttgaactgatccgcaaaggatatacacaacagttggcttttcgccaaaagtccagcgcatatgcagcatttcaataccgcccaccatctacttggttgaccgcttatgttgttaaggttttcgctctcgcagcaaatctgattgcaatcgattctaaggatttgtgcgagactgttaaatggttaatcttggagaaacagaaacctgacgggatctttcaggaagatggtcctgttatccaccaggagatgatcgggggatttagagatacacgtgaaaaagacgtttctctgactgcattcgtcttgatcgctttgcacgaggccaaggacatctgtgaggcccaagttaatagtttgggtagatccattgcaaaagccggggactttctggaaaatcactatagggaactgagacgcccttacacagtagcaatcgcagcatacgctttggcactgctcggaaaactcgaaggggacagattaacaaagtttttgaataccgctaaggaaaaaaacagatgggaagagccaaatcagaaactgtacaacgtagaagctactagttatgctttgctcgccttgttggccagaaaagattacgatacaaccccacctgtagtaagatggctgaatgagcaaaggtattacgggggaggatacggaagcactcaagcaaccttcatggtttttcaagcactcgcacagtaccaaaaggacgttcctgaccacaaagagttgaacttggacgtcagcattcagctccca

SEQ ID NO: 21 - DNA, 900 bp, Artificial Sequence
  Nucleotide sequence of first variant Sus sp. C3d
cacctgattcaaacgccatcagggtgcggagagcaaaacatgattggtatgaccccaacggtgatcgcagtccactatctcgattcaacggaacagtgggagaaattcggattagagaaaagacaagaagcgctcgaattgatcaaaaagggctatacgcagcagttagcttttagacagaagaattccgcgttcgctgcgttccaagacagacttagttcaacatggttaactgcgtatgttgtgaaagttttcgccatggctgcgaatctgattgcgatcgattcacaagtgttatgtggcgctgtgaaatggttaattcttgaaaagcaaaagccagatggcgtgtttgaggaaaatggcccagtcattcaccaggaaatgatcggcggatttaagaatacggaggaaaaagatgtatcattaacggcatttgtcttaattgcacttcaagaggcgaaggacatttgtgagcctcaggtgaacagtttattaagaagtattaacaaggcgcgtgactttctggcggattactatctcgagttgaagaggccttatacggtagctatcgcgggatacgcactcgccttgtcagataagttagatgaaccatttctgaataaattactttcaacggcaaaagagagaaatcgttgggaagaacctggacaaaagttatataacgtagaggcaacgagttacgcacttcttgccctcttagtggttaaggacttcgacagcgtacctccaattgtcagatggctgaacgagcaacgctactatgggggagggtacggcagcacgcaggcaacgtttatggtcttccaggcgttagcgcagtaccaaaaggacgtaccagatcacaaagatttaaacctcgacgtcagtatccacttacca

SEQ ID NO: 22 - DNA, 900 bp, Artificial Sequence
  Nucleotide sequence of second variant Sus sp. C3d
cacttaatccagactcctagtggatgtggcgaacagaatatgatcgggatgacacctaccgtaattgcggttcactacttagacagtacagagcaatgggaaaagtttgggctcgaaaagcgccaggaggctcttgagttaattaagaaaggttacacacaacaactcgcattcaggcaaaaaaacagtgcatttgcggcatttcaggatcgcttaagcagtacgtggctcaccgcatacgtcgtaaaggtgtttgcgatggccgcaaacttaatcgcaattgacagtcaggtactgtgcggagcggttaagtggcttatcttagagaaacagaaacctgacggggtattcgaagagaacggtcctgttatccaccaagagatgattgggggtttcaaaaacaccgaagagaaggacgttagtctcaccgctttcgtgctgatcgccttacaggaagccaaagatatctgcgaaccacaagtaaattcactccttcgttcaatcaataaagccagggatttcttagccgactattacttggaactcaaacgtccatacaccgttgcgattgccgggtatgctttagcgctcagcgacaaactcgacgagcctttcttaaacaagctgttaagtaccgctaaggaacgcaacaggtgggaggagccaggtcagaaactttacaatgttgaagctacctcatatgctttattagcgttactcgtcgtaaaagattttgattcagtgccacctatcgtacgttggttaaatgaacagaggtattacggtggcggatatgggtcaacacaagcgaccttcatggtatttcaagcactcgcacaatatcagaaagatgtgcctgaccacaaggacctgaatttagatgtatcaattcaccttcct

SEQ ID NO: 23 - DNA, 900 bp, Artificial Sequence
  Nucleotide sequence of first variant Canis familiaris C3d
cacttaatcgtcactccaagtggatgcggcgaacagaatatgatcggaatgacaccaacggtaattgcgctccactatttagaccagactgagcaatgggataagtttggactcgaaaagaggcaggaagcgctcgagttaatcaaaaagggctatacgcagcagttagcgtttaggcagccaaattccgcgtttgcagcgttccaaaacagaccatcatcaacgtggcttacggcttatgtcgttaaagtgttctcattagcgactaatcttattgcaatcgaggctcaggtcctttgtggcgcggttaaatggttaattctcgaaaagcaaaaaccagacggcattttccaagaggacggccctgtaattcaccaagaaatgacgggcggttttcgcgaagcggaagagaaatcagtaagtcttaccgcgttcgtgctgattgcgttgaaagaagccgaagacatttgcattgggcaggtcaatatcctccctagttcaattgaaaaagctggcaactatctggccgcacactatcagaatctccgtaggccttatagcgtcgcaatagccggttacgcattagcacacctcgataaattagagggtgacaacttacgcaagtttctcaatacggcaagggatagaaatcgttgggtagaaccagggaagaaactttataatgtagaggcgacaagttacgcgctgctcgctcttttattactaaaggacttcgacaacgtcccacctgtggtgaggtggcttaatgaacagcgttactatggcggaggatacggatcaacgcaagctacattcatggtctttcaagcgctcgcgcaatatcagaaagatgtgccaaatcacaaggatctcaatttacaagtaagtatcaatctccca

SEQ ID NO: 24 - DNA, 900 bp, Artificial Sequence
  Nucleotide sequence of second variant Canis familiaris C3d
cacctgattgtaacgcctagcggttgtggagagcaaaacatgattgggatgacccctactgtgatcgcattacactacctcgatcaaacagaacagtgggacaaattcggtttagagaaacgtcaagaggctttagaactgattaagaaaggttacacccaacaactcgctttccgtcaacctaacagtgctttcgcggcttttcagaatcgtcctagtagtacatggttaaccgcatacgtagtgaaggtatttagtcttgcaacgaacttaatcgctatcgaagcgcaagtgttgtgcggagccgtgaagtggctcatcttagagaaacagaagcctgatggtatctttcaggaagatggaccagtcatccaccaggagatgactggagggttcagagaggccgaggaaaagagcgtgtcattgacagcttttgtcttaatcgccctcaaggaggcggaggatatctgtatcggccaagtaaacattttaccatcatctatcgagaaggccggtaattacttagctgcgcactaccaaaacttaagacgcccatactcagttgcgattgcagggtatgcgctcgcgcacttggacaagctcgaaggcgataatctgaggaaattcttaaacactgcccgtgaccgcaacagatgggtcgagccaggaaaaaagttgtacaacgtcgaagctacctcatatgcattacttgccttactccttttgaaagattttgataatgtacctccagtagttcgttggttgaacgagcaaaggtattacgggggcggttatggtagtactcaggcgacgtttatggtattccaggcattagcacagtaccaaaaggacgtacctaaccacaaagacttaaaccttcaggtctcaattaacttacct

SEQ ID NO: 25 - DNA, 888 bp, Macaca mulatta
accccctcgggctgcggagaacagaacatgatcaccatgacgcccacagtcatcgctgtgcattacctggatgaaacggaacagtgggagaagttcggcccggagaagcggcagggggccttggagctcatcaagaaggggtacacccagcagctggccttcagacaacccagctctgcctttgcggccttcctgaaccgggcacccagcacctggctgaccgcctacgtggtcaaggtcttctctctggctgtcaacctcattgccatcgactcccaggtcctctgcggggctgttaaatggctgatcctggagaagcagaagcccgacggggtcttccaggaggatgcgcccgtgatacatcaagaaatgactggtggattccggaacaccaacgagaaagacatggccctcacggcctttgttctcatctcgctgcaagaggctaaagagatttgcgaggagcaggtcaacagcctgcccggcagcatcactaaagcaggagacttccttgaagccaactacatgaacctacagagatcctacactgtggccatcgctgcctatgccctggcccagatgggcaggctgaagggacctcttctcaacaaatttctgaccacagccaaagataagaaccgctgggaggagcctggtcagcagctctacaatgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggatgtccctgatcacaaggaactgaacctggatgtgtccctccaactgccc

SEQ ID NO: 26 - DNA, 888 bp, Artificial Sequence
  First variant Macaca mulatta C3d
acgccaagcggatcaggcgagcagaatatgatcactatgacaccaaccgtaattgcggtccattatctcgacgaaaccgaacagtgggaaaaatttgggccggaaaagcgtcaaggcgctctcgagttgatcaagaaaggctacacgcaacagttagcgttccgtcaaccatcatcagcgttcgccgctttcctgaatcgtgcgccatcaacgtggctcacagcgtatgtagtgaaggtatttagcctcgccgtaaatttaatcgcgattgacagtcaagtgttatgcggcgcggtcaagtggctcattcttgaaaagcaaaagccagatggcgtattccaagaggacgccccagtcatccaccaagagatgacaggcggctttcgcaatactaatgagaaggacatggcgttaaccgcgtttgtcttaatcagtttacaggaagccaaagaaatttgtgaggaacaggtaaatagtttacctgggagtattacgaaagcgggcgatttcttagaagcaaattacatgaatctccaacgctcatacacggtagcgatcgcggcttatgccttagcgcagatggggagattaaaaggcccattactgaacaagtttttaacaaccgcaaaagacaagaataggtgggaggaaccaggccaacaactttataacgtcgaagcaacgtcatacgcattattagcactcttacaactcaaggacttcgacttcgtaccacctgtggtacggtggcttaacgaacaaaggtattacgggggcggatacggcagcacgcaagcgactttcatggtctttcaagcactcgcacagtaccagaaggatgttcctgatcacaaggaattaaacttagatgtcagtctgcagttacca

SEQ ID NO: 27 - DNA, 888 bp, Artificial Sequence
  Second variant Macaca mulatta C3d
actccttcagggagtggagaacaaaacatgattacaatgacccctacagtgatcgccgtacactacttagatgagacagagcaatgggagaaattcggtcccgagaaaagacagggagcgttagaacttattaaaaagggatatacacagcaactcgcttttaggcagcctagtagcgcatttgctgcgtttctcaacagagcccctagtacatggttaacggcttacgtcgtaaaagtgttctcattagcggtgaacctgattgcaatcgattcgcaggtactgtgtggagccgtgaaatggttaatcttagagaaacagaaacctgacggagtgtttcaggaagatgcacctgtaattcaccaggaaatgaccgggggcttcagaaacacaaacgaaaaagatatggctctgacagctttcgtgctgatttccctccaagaggcgaaggagatctgcgaagagcaagtgaactcactcccaggatcaatcaccaaggccggggactttctggaggcgaactatatgaacttgcagaggagctataccgtcgcaattgccgcatacgcgctcgcacaaatgggacgtctcaaaggacctctgttaaataaattcctcacgacggcgaaggataaaaaccgatgggaagaacctgggcaacagttgtacaatgtagaggcgaccagttatgcgctgctcgcgttgctccagttgaaagattttgattttgtccctccagtagtcagatggttgaatgagcagcgttactatggaggggggtatggatcaacacaggcaacgtttatggtattccaggcgttagcgcaatatcaaaaagacgtgccagaccacaaagagcttaatctcgacgtatcattacaactccct

SEQ ID NO: 28 - DNA, 888 bp, Artificial Sequence
  Third variant Macaca mulatta C3d
acaccgtctggtagcggtgagcaaaatatgataaccatgactccgactgttatagcagttcactatttagacgagactgaacaatgggaaaagtttggaccggaaaaaaggcaaggtgcactggaattaataaaaaaaggttatacgcagcaactagcgttcaggcagccgtccagcgctttcgcagcatttctgaacagggctccgtccacttggttgacggcatatgtcgtgaaagtttttagtttggcagttaacttgatagcgatcgatagccaggttttgtgtggtgcagtaaagtggttgatactcgaaaagcaaaagccggatggtgtttttcaagaagacgccccggttatccatcaggagatgactggaggtttcaggaataccaatgaaaaggatatggcattgactgcattcgtattgataagcttgcaagaagcaaaggagatatgtgaagaacaagttaattccttgccgggttccataacaaaggctggtgattttctcgaggctaattatatgaatctgcaacgaagttatacagttgctatagcagcctacgcactcgctcaaatgggtcgcttgaagggtccgctcctgaataagttcttgactactgctaaggacaaaaatagatgggaagagccgggacagcaactgtataatgttgaagctactagctacgctttgctggctctgttgcaactgaaggatttcgatttcgttcccccggttgttaggtggttaaacgagcaacgctattatggcggaggttacgggtcgactcaagctacatttatggtttttcaggctctggcccagtatcagaaagatgtccccgatcataaggagctcaatctggacgttagcttgcagttgccg

SEQ ID NO: 29 - protein, 296 aa, Macaca mulatta
TPSGSGEQNMITMTPTVIAVHYLDETEQWEKFGPEKRQGALELIKKGYTQQLAFRQPSSAFAAFLNRAPSTWLTAYVVKVFSLAVNLIAIDSQVLCGAVKWLILEKQKPDGVFQEDAPVIHQEMTGGFRNTNEKDMALTAFVLISLQEAKEICEEQVNSLPGSITKAGDFLEANYMNLQRSYTVAIAAYALAQMGRLKGPLLNKFLTTAKDKNRWEEPGQQLYNVEATSYALLALLQLKDFDFVPPVVRWLNEQRYYGGGYGSTQATFMVFQALAQYQKDVPDHKELNLDVSLQLP

SEQ ID NO: 30 - protein, 296 aa, Macaca mulatta; identical to SEQ ID NO: 29